(19) United States
(12) Patent Application Publication
(10) Pub. No.: US 2011/0229556 A1
(43) Pub. Date: Sep. 22, 2011

(54) LIPID-COATED POLYMER PARTICLES FOR IMMUNE STIMULATION

(75) Inventors: Darrell J. Irvine, Arlington, MA (US); Anna Bershteyn, Seattle, WA (US); Jaehyun Moon, Cambridge, MA (US)

(73) Assignee: Massachusetts Institute of Technology, Cambridge, MA (US)

(21) Appl. No.: 13/053,101

(22) Filed: Mar. 21, 2011

Related U.S. Application Data
(60) Provisional application No. 61/315,485, filed on Mar. 19, 2010.

(51) Int. Cl. A61K 9/127; A61K 39/385; A61P 37/04; B82Y 5/00

(52) U.S. Cl. 424/450; 424/193.1; 977/773; 977/799; 977/907

(57) ABSTRACT

The invention provides delivery systems comprised of lipid coated polymer core particles, as well as compositions, methods of synthesis, and methods of use thereof. The particles can be used to carry antigen and adjuvant, resulting in enhanced immune responses.

### RELATED APPLICATIONS

This application claims the benefit under 35 U.S.C. §119(e) of U.S. Provisional Application Ser. No. 61/315,485, entitled “MICRO- AND NANOPARTICLES WITH SELF-ASSEMBLED LIPID COATINGS FOR SURFACE DISPLAY OF DRUGS SUCH AS VACCINE ANTIGENS AND ADJUVANTS” filed on Mar. 19, 2010, which is incorporated by reference herein in its entirety.

### FEDERALLY SPONSORED RESEARCH

This invention was made with Government support under Grant No. R21AI073165 from the NIH and Grant No. BES0348259 from the NSF. The Government has certain rights in the invention.

### BACKGROUND OF INVENTION

The immune system has evolved to respond strongly to antigens encountered in micro- or nano-particulate form, likely reflecting the intrinsic particulate nature of foreign microbes. B-lymphocytes are strongly activated by particles displaying repeat copies of antigens capable of crosslinking B-cell receptors [1-3], and particulate delivery also allows antigens to be processed and loaded onto class I MHC molecules, enhancing CD8⁺ T-cell responses [4-6]. These findings, combined with the desire to control the duration of exposure to antigen via controlled release, have motivated extensive studies of biodegradable polymer micro- or nano-particles as potential vaccine delivery materials [7-19]. These technologies have failed to move into the clinic in part due to the challenges of low antigen encapsulation efficiency and denaturation of protein antigen during the encapsulation process [8, 10].

### SUMMARY OF INVENTION

The invention is premised in part on a novel and inventive delivery system for stimulating antigen-specific immune responses. This delivery system comprises particles comprising antigen or antigen and adjuvant. As described in greater detail herein, these particles were unexpectedly able to elicit an antibody response against nanogram doses of antigen, in some instances with only a single immunization, and in still other instances for extended periods of time. The robust immune responses observed at such low antigen doses were surprising and unprecedented, and far superior to those observed using the same dose of antigen in solution or the same dose of antigen formulated with traditional adjuvants such as Freund's adjuvant and alum (the only adjuvant currently licensed for use in the United States). The particles of the invention therefore were able to effect robust antigen specific immune responses using antigen doses that are on the order of 1000-fold lower than the antigen doses typically used in soluble protein immunizations.

Moreover, simultaneous display of antigens and adjuvants on the same particles again elicited unexpectedly robust antigen specific immune responses, including antibody (or humoral) responses. In particular, it was found according to the invention that, when monophosphoryl lipid A (MPLA) was used as the adjuvant, the immune response was prolonged, lasting for at least 12 weeks. In comparison, when alpha-galactosylceramide (αGC) was used as the adjuvant, much higher antibody titres were achieved in the short term (as compared to particles comprising antigen alone).

The invention therefore provides, inter alia, unexpected antigen and/or adjuvant dose-sparing compositions and methods. Lipid-coated microparticles and nanoparticles of the invention may be used to greatly reduce the dose of antigen necessary to achieve immunity. This may lower the cost of vaccine manufacture and reduce the risk of seasonal or pandemic vaccine shortages.

Thus, in one aspect, the invention provides a particle or a population (or plurality) of particles each particle comprising a biodegradable polymer core, a lipid bilayer coat, wherein the lipid bilayer coat is conjugated to an antigen, and an adjuvant incorporated into the lipid bilayer coat. The particle may be a nano- or microparticle. The population of particles may comprise nanoparticles and microparticles.

In some embodiments, the adjuvant is a lipid-like adjuvant. In some embodiments, the adjuvant is MPLA. In some embodiments, the adjuvant is αGC. In some embodiments, the particles comprise two adjuvants, such as but not limited to MPLA and αGC.

In another aspect, the invention provides a particle comprising a PLGA polymer core, a lipid bilayer coat comprising phosphocholine, phosphoglycerol and phosphoethanolamine, wherein the lipid bilayer coat is conjugated to an antigen, and MPLA incorporated into the lipid bilayer coat.

In another aspect, the invention provides a particle comprising a PLGA polymer core, a lipid bilayer coat comprising phosphocholine, phosphoglycerol and phosphoethanolamine, wherein the lipid bilayer coat is conjugated to an antigen, and αGC incorporated into the lipid bilayer coat.

In another aspect, the invention provides a particle comprising a PLGA polymer core, a lipid bilayer coat comprising phosphocholine, phosphoglycerol and phosphoethanolamine, wherein the lipid bilayer coat is conjugated to an antigen, and adjuvant Pam3Cys incorporated into the lipid bilayer coat.

In another aspect, the invention provides a particle comprising a polymer core, a lipid bilayer coat comprising phosphocholine, phosphoglycerol and phosphoethanolamine, wherein the lipid bilayer coat is conjugated to an antigen, and one or more adjuvants selected from the group consisting of MPLA, αGC and Pam3Cys incorporated into the lipid bilayer coat.

Various embodiments apply equally to the various aspects of the invention. Some of these embodiments follow.

In some embodiments, the biodegradable polymer is PLGA.

In some embodiments, the particle and more particularly the lipid bilayer coat comprises a functionalized component of a lipid bilayer, such as a functionalized lipid. In some embodiments, the particle and more particularly the lipid bilayer coat comprises a maleimide functionalized lipid.

In some embodiments, the particle and more particularly the lipid bilayer coat comprises phosphocholine. In some embodiments, the particle and more particularly the lipid bilayer coat comprises phosphoglycerol. In some embodiments, the particle and more particularly the lipid bilayer coat comprises a phosphoethanolamine. In some embodiments, some phosphoethanolamine is functionalized with a reactive group. In some embodiments, the reactive group is maleimide. In some embodiments, the functionalized lipid is a maleimide functionalized phosphoethanolamine.

In some embodiments, the antigen is conjugated to a functionalized lipid in the lipid bilayer coat. In some embodiments, the antigen is a protein antigen. In some embodiments, the antigen is a whole protein antigen. In some embodiments, the antigen is a peptide antigen. In some embodiments, the antigen is a polysaccharide.

In another aspect, the invention provides a composition comprising any of the foregoing particles or particle populations and a pharmaceutically acceptable carrier.

In another aspect, the invention provides a composition comprising any of the foregoing particles or particle populations and a cryopreservant or a lyopreservant or an excipient suitable for lyophilization. The lyopreservant may be but is not limited to sucrose.

In another aspect, the invention provides a pharmaceutical composition or formulation comprising any of the foregoing particles or particle populations, wherein the antigen is present in a dose of 1-10 nanograms, or 2 to 10 nanograms.

In another aspect, the invention provides a pharmaceutical composition or formulation comprising particles comprising a biodegradable polymer core, a lipid bilayer coat, wherein the lipid bilayer coat is conjugated to an antigen at the external (or outermost surface of the lipid bilayer), and a pharmaceutically acceptable carrier, wherein the antigen is present in a dose of 1-10 nanograms.

In some embodiments, the pharmaceutical composition further comprises an adjuvant incorporated into the lipid bilayer coat. In some embodiments, the adjuvant is MPLA. In some embodiments, the adjuvant is Pam3Cys. In some embodiments, the adjuvant is αGC. In some embodiments, the adjuvant is two adjuvants, such as MPLA and αGC.

In one aspect, the invention provides a method comprising administering to a subject in need of immune stimulation a composition comprising any of the foregoing particles or compositions (including pharmaceutical compositions) in an effective amount to stimulate an antigen specific immune response.

In some embodiments, the subject has or is at risk of developing an infection. In some embodiments, the subject has or is at risk of developing a cancer.

In some embodiments, the effective amount is 2.5-10 nanograms of antigen.

In some embodiments, the antigen specific immune response is a humoral (antibody) immune response. In some embodiments, the antigen specific immune response is a cellular immune response. In some embodiments, the antigen specific immune response is a humoral and a cellular immune response.

In some embodiments, the subject receives a single administration of antigen. In some embodiments, the particles are nanoparticles.

In some embodiments, the subject receives two administrations of antigen.

In some embodiments, the immune response lasts for more than a month, more than two months, or more than three months.

In another aspect, the invention provides a method comprising contacting cells with any of the foregoing particles or compositions in an effective amount. In some embodiments, the cells are contacted in vitro. In some embodiments, the cells are antigen presenting cells. In some embodiments, the cells are dendritic cells.

In another aspect, the invention provides a method comprising conjugating an antigen to a functionalized lipid of a particle comprising a biodegradable polymer core and a lipid bilayer coat, wherein the lipid bilayer coat comprises an adjuvant.

In another aspect, the invention provides a method comprising conjugating an antigen to a functionalized lipid of a particle comprising a biodegradable polymer core and a lipid bilayer coat, and incorporating into the lipid bilayer coat an adjuvant.

In some embodiments, the adjuvant is a lipophilic adjuvant. In some embodiments, the adjuvant is MPLA. In some embodiments, the adjuvant is αGC. In some embodiments, the adjuvant is two adjuvants, such as MPLA and αGC.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF DRAWINGS

FIG. 1A. Schematic of lipid-coated micro-/nano-particles with surface-displayed antigen.

FIG. 1B. Synthesis of lipid-enveloped micro- or nano-particles with surface-displayed antigen and molecular adjuvants. (i) Light scattering analysis of purified particle size distributions for microparticles (dashed line) or nanoparticles (solid line) synthesized by homogenization or sonication, respectively, to disperse lipid/polymer emulsion during particle synthesis. (ii) Confocal imaging of lipid-enveloped microparticles bearing ˜7×10⁴ green fluorescent protein molecules per particle (green, GFP intrinsic fluorescence). (iii) Confocal imaging of microparticles modified with rhodamine-labeled Pam3Cys (red fluorescence, lipid-like TLR-2 agonist) incorporated via post-insertion or through self-assembly during particle synthesis.

FIG. 2. Priming of naïve CD4⁺ or CD8⁺ T-cells by antigen-conjugated lipid-enveloped particles. Primary splenic DCs were incubated with OVA-conjugated microparticles (with or without post-inserted MPLA) for 3 hrs, then co-cultured with naïve CFSE-labeled OT-I (CD8⁺) or OT-II (CD4⁺) T-cells. Proliferation of T-cells was assessed after 3 days by flow cytometry. Shown are representative flow histograms (10:1 particle:DC ratio) and mean percentages of proliferated cells from triplicate wells (±St. dev.). The maximum particle:DC ratio (40:1) corresponds to a total dose of 2.6 ng OVA in the 200 μL culture.

FIG. 3. Serum IgG responses to particle-delivered or soluble OVA at a modest but conventional dose of 0.5 μg OVA. BALB/c mice were immunized s.c. with 500 ng of OVA in solution or displayed on lipid-coated microparticles and boosted on day 21 with the same formulations. Shown are analyses of sera collected on day 28: (A) Total anti-OVA IgG ELISA on serum from mice immunized with OVA-particles (dotted lines) or OVA solution (solid lines); (B) Endpoint total IgG titers (**, P<0.01) (C) total OVA-specific IgG concentration in sera (***, P<0.0001).

FIG. 4. Serum IgG responses elicited by lipid-coated particles vs. conventional adjuvants at limiting antigen doses. Groups of BALB/c mice (n=4) were immunized s.c. with 10 ng OVA displayed on microparticles (“MP”), dissolved in saline (“soln”), mixed with alum, or mixed with MPLA and αGC; animals were boosted on day 21 with the same formulations. In both the particle-displayed and soluble adjuvant cases, equimolar quantities of 1.3 μg MPLA and 600 ng αGC were used. (A) Post-boost peak (day 28) and late (day 105) endpoint titers from individual mice. (B) Mean endpoint titers (±SEM) for particle immunizations over time (**, P=0.0053). (C, D) Endpoint IgG₁ (C) and IgG_{2A} titers at day 28. (E, F) Avidity of OVA-specific IgG in each group measured at day 28 for all groups (E) or for the particle-immunized groups over time (F). (N.B.: No binding detected. *, **, *** in panels A, C-E: P<0.05 relative to soln+MPLA/αGC, soln, or alum at the same time point, respectively).

FIG. 5. IgG responses following dose sparing immunizations with lipid-coated particle immunogens. Groups of C57B1/6 mice (n=3) were immunized with lipid-coated microparticles delivering the indicated dose of OVA and boosted on day 14. The particle-only conditions (black circles) carried OVA alone; otherwise, 13 μg MPLA or 6 μg αGC were added via the post-insertion method to the antigen-loaded particles. Shown are mean endpoint titers (±SEM) for dose titrations of particles carrying OVA and (A) MPLA or (B) αGC. (C) Groups of BALB/c mice (n=4) were immunized with diminishing doses of OVA co-displayed with 1.3 μg MPLA and boosted on day 14 to determine the minimum dose capable of eliciting measurable antibody responses. Post-boost peak (day 28) endpoint titers are shown for individual mice (*, P<0.05).

FIG. 6. Comparison of adjuvant effect of lipid-enveloped microparticles vs. nanoparticles. Groups of BALB/c mice (n=3) were immunized s.c. with 10 ng OVA displayed on microparticles (“MP”) or nanoparticles (“NP”), and boosted on day 14. For comparison, particles from the same syntheses were loaded with 1.3 μg MPLA and 600 ng αGC via the post-insertion method. Bars show mean endpoint titers±SEM. N.D., no antigen-specific IgG detected above background (*, P<0.001 vs. MP; **, P<0.01 vs. MP; ⋄, P<0.01 vs. NP; †, P<0.05 vs. NP; all comparisons made by Bonferroni post-tests at the same time point).

FIG. 7. Analysis of synergy between MPLA and αGC in particle immune responses. Groups of BALB/c mice (n=4) were immunized s.c. with OVA displayed on microparticles and boosted on day 21; endpoint total IgG titers were determined and shown are means±SEM. (A) Mice were immunized with 25 ng OVA and 1.3 μg MPLA and/or 600 ng αGC co-loaded onto microparticles. (B) Mice were immunized with 10 ng OVA-conjugated nanoparticles co-loaded with MPLA, αGC, or both adjuvants, and compared to mice given the same doses of the adjuvant molecules injected in soluble form 10 min before injection of the antigen-loaded particles at the same site. Titers were assessed on day 28 (*, P<0.05 vs. soln MPLA; **, P<0.05 vs. soln αGC). (C, D) Mice were immunized with microparticles displaying 10 ng OVA, followed 10 minutes later by microparticles displaying 1.3 μg MPLA and 600 ng αGC injected at the same site (C, “separate”). For comparison, mice received an equivalent number of blank microparticles, followed 10 minutes later by microparticles co-displaying 10 ng OVA, 1.3 μg MPLA, and 600 ng αGC(C, “together”). *, P=0.0284; **, P=0.0070.

FIG. 8. Dose sparing of molecular adjuvants by lipid-coated particles. Groups of BALB/c mice (n=4) were immunized s.c. with 2 ng OVA displayed on microparticles co-loaded with the indicated quantities of αGC via the post-insertion method (αGC on particles). A second group of mice was immunized by injecting the indicated doses of αGC followed 10 min later by 2 ng OVA-microparticles at the same site (αGC solution). Shown are endpoint total IgG titers for individual mice two weeks after a single immunization (*, P<0.05).

### DETAILED DESCRIPTION OF INVENTION

The invention provides delivery systems for antigens and adjuvants having surprising and unexpected increased potency. The delivery systems of the invention comprise particles having at a minimum a polymer core, a lipid bilayer coating the polymer core (i.e., a lipid bilayer coat), and an antigen conjugated to the external surface of the lipid bilayer. In some instances, the particles further comprise one or more adjuvants. The adjuvants may be conjugated to the external surface of the lipid bilayer or they may be inserted or incorporated into the lipid bilayer during or post synthesis of the lipid bilayer. The particles may or may not also comprise additional agents, for example in their core. Particles of the invention that display antigen or antigen and adjuvant were found to stimulate surprisingly robust immune responses even when extremely low doses of antigen were administered to a subject, including when such low doses were administered only once to the subject. The increased potency of these particles also results in lower doses of adjuvant being administered to subjects. This can then lower the incidence and/or severity of adverse side effects of various adjuvants. Accordingly, the particles of the invention are novel and inventive delivery vehicles that find use in situations where antigen supplies are limited, where antigens are poorly immunogenic, where there is a need for rapid immune response induction (including rapid IgG immune responses), where patient compliance throughout a more traditional multi-immunization protocol is required, and/or where it is desirable to reduce side effects that occur when higher doses of adjuvant are required.

As an example, and as described in greater detail herein, a strong class-switched, high avidity humoral immune responses may be elicited by particles of the invention comprising, for example, a biodegradable polymer (e.g., poly(lactide-co-glycolide)) core enveloped by a lipid bilayer (e.g., a PEGylated phospholipid bilayer), with antigens (e.g., protein antigens) covalently anchored to the lipid surface and lipophilic adjuvants inserted in the bilayer coating. As described in the Examples, surprisingly, these particles elicited high endpoint antigen-specific IgG titers (>10⁶) that were sustained for over 100 days after two immunizations with as little as 2.5 ng of antigen. Strong antigen specific titres were also detected after a single immunization with only 10 nanograms of surface displayed protein antigen co-delivered with adjuvants such as MPLA or αGC. Particles displaying protein without adjuvant elicited higher titres than adjuvant co-dissolved with protein in saline solution. MPLA provided the highest sustained IgG titers at these ultra-low antigen doses, while αGC promoted a rapid rise in serum IgG after one immunization, which may be valuable in acute care setting such as disease pandemics. The dose of αGC required to boost the antibody response was also spared by the use of particles as the delivery vehicle. It was also found in accordance with the invention that MPLA and αGC do not act synergistically when displayed together on lipid-coated particles, in contrast to their reported behavior when used in solution (Salio et al PNAS 104(51) 20490-20495 (2007)).

Co-display of antigen and adjuvant on the same particle promotes stronger antibody responses than display of antigen or adjuvant on separate particles delivered at the same injection site, suggesting that a single particle displaying multivalent antigen and adjuvant on a lipid shell, much like a lipid-enveloped virus or bacterium, induces a stronger immune response than a traditional antigen and adjuvant formulations.

The ability to administer antigen and/or adjuvant at low doses is useful for a number of reasons. First, dose sparing of antigen is of significant interest in the setting of seasonal influenza vaccines, where production issues have in the past led to vaccine shortages, as well as in bioterrorism and pandemic vaccine development settings, where rapid deployment of limited vaccine stocks may be critical [29-34]. Second, dose sparing of adjuvants such as MPLA and αGC lowers the likelihood of reactogenicity or systemic side effects that can block clinical translation of promising adjuvant candidates for prophylactic vaccines [35]. Lastly, dose titration is a powerful strategy for comparing potency of candidate vaccines in mice, allowing important differences in vaccine potency to be revealed that may be missed by immunizations with high antigen doses [36]. These quantitative features of vaccination are infrequently characterized in small-animal models but may be relevant for predicting the performance of candidate particle-based vaccines in non-human primates and humans.

### Particles

As used herein, a particle of the invention is a particle comprising a polymer core and a lipid bilayer coat. The particles are therefore not liposomes or vesicles both of which are classically defined as having a void volume and/or an aqueous fluid environment at their core. The particles are synthetic and not naturally occurring. They are not viruses or virus fragments, although their structure may be referred to as a mimic of a virus or other naturally occurring delivery system.

The particles may be nanoparticles or microparticles. The terms are used to denote the size of the particles, typically characterized by particle diameter.

As used herein, nanoparticle refers to any particle having an average diameter in the range of 1 to less than 1000 nanometers. In some instances, such particles will have an average diameter in the range of 50 to 900 nanometers, 50 to 800 nanometers, 50 to 700 nanometers, 50 to 600 nanometers, 50 to 500 nanometers, 50 to 400 nanometers, 50 to 300 nanometers, 50 to 250 nanometers, 50 to 225 nanometers, 50 to 200 nanometers, 50 to 150 nanometers, 50 to 125 nanometers, 50 to 100 nanometers, and/or 100 to 200 nanometers. The lower end of these ranges may alternatively be about 100 nanometers.

As used herein, microparticle refers to any particle having an average diameter in the range of 1 to less than 1000 micrometers. In some instances, such particles will have an average diameter in the range of 1 to 500 micrometers, 1 to 100 micrometers, 1-50 micrometers, 1 to 25 micrometers, 1-20 micrometers, 1-15 micrometers, 1-10 micrometers, 1-5 micrometers, or 1-3 micrometers.

The particles may be of any shape and are not limited to a perfectly spherical shape. As an example, they may be oval or oblong. As a result, particle size is referred to in terms of average diameter. As used herein, average diameter refers to the average of two or more diameter measurements. The dimensions of the particles may also be expressed in terms of the longest diameter or cross-section.

The particles may be synthesized or modified post-synthesis to comprise one or more reactive groups on their external (or outermost) surface for reaction with reactive groups on agents such as antigens and adjuvants. These reactive groups include without limitation thiol-reactive maleimide head groups, haloacetyl (e.g., iodoacetyl) groups, imidoester groups, N-hydroxysuccinimide esters, pyridyl disulfide groups, and the like.

The particles may be isolated, intending that they are physically separated in whole or in part from the environment in which they are synthesized. As an example, particles comprising an agent (i.e., their “cargo” or “payload”) may be separated in whole or in part from particles lacking agent. As another example, particles may also be separated from liposomes that do not comprise a polymer core. Separation may occur based on weight (or mass), density (including buoyant density), size, color and the like (e.g., where the cargo of the particle is detectable by its energy emission), etc. Moreover, nanoparticles may be separated from microparticles using for example centrifugation.

The particles are not conjugated to cells and are not administered with cells to a subject. Instead the particles may be administered alone or with other agents, typically in a pharmaceutically acceptable carrier. They may or may not comprise a targeting agent. The particles may support controlled release of agents including small molecule drugs from their polymer core.

### Synthesis Methods

The following is a general synthesis strategy for the lipid-coated particles of the invention as well as an exemplary detailed synthesis strategy. The particles of the invention have a polymer core and a lipid bilayer coat (i.e., a lipid bilayer that surrounds the polymer core). The core is preferably comprised of one or more biodegradable polymers or copolymers such as but not limited to PLGA. The lipid bilayer is constructed around the polymer core by self-assembly during emulsion synthesis, in which the lipid acts as a surfactant to stabilize the oil-water interface of the emulsion.

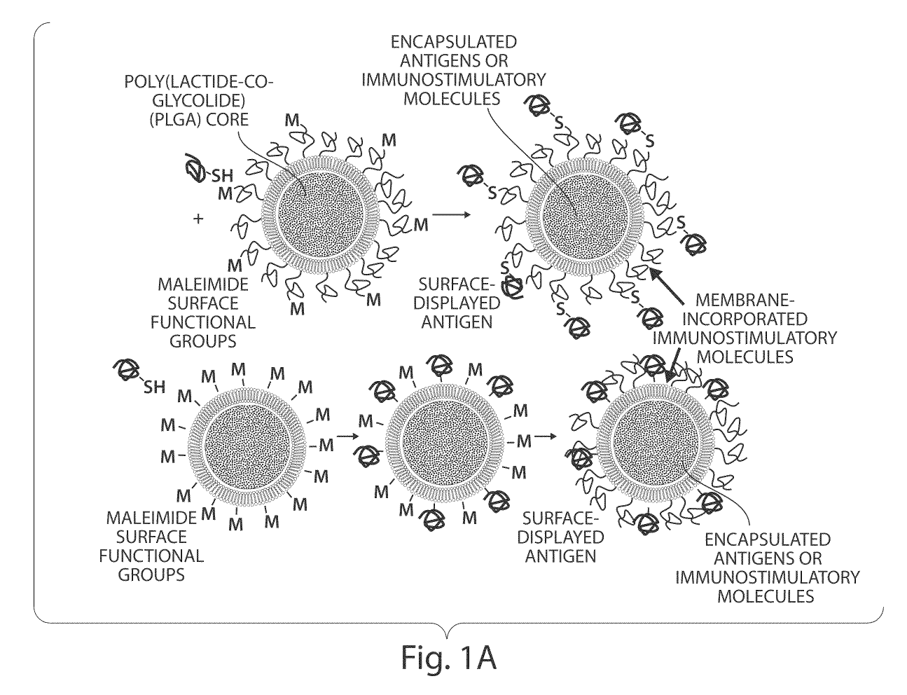

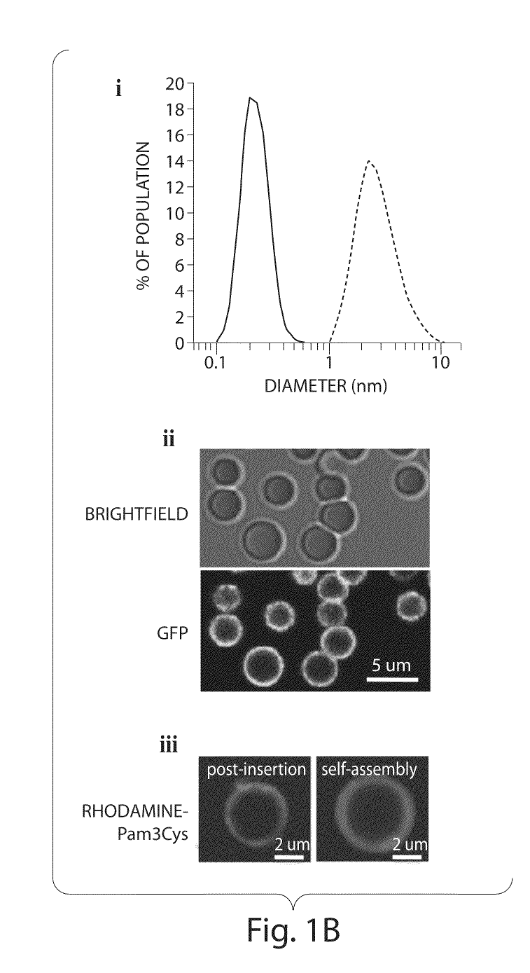

Antigen may be present within the particle although more preferably it is present on the surface of the particle. This may be achieved by conjugating antigen such as protein, peptide, or polysaccharide antigen to the surface of the particle, as schematically illustrated in FIG. 1A. To further enhance the potency and duration of antigen specific immune responses such as antibody responses, the fluid lipid bilayer coat may further comprise adjuvants. Such adjuvants may be incorporated into the lipid bilayer during or after synthesis. In some instances, the adjuvants may be tethered to the particles.

Lipid-like (or lipophilic) molecules, including lipid-like (or lipophilic) adjuvants, spontaneously integrate into the fluid lipid bilayer coat, and in doing so further enhance and prolong immune responses to unprecedented low antigen doses. Examples of lipid-like adjuvants include TLR 2 and TLR 4 agonists and invariant natural killer T cells (iNKT) agonists. Specific examples include the TLR4 agonist Monophosphoryl Lipid A (MPLA) which is a lipopolysaccharide-like molecule approved for vaccine applications in Europe (Mata-Haro et al. Science 316(5831), 1628-1632 (2007)) and in the CERVARIX™ vaccine in the U.S., the TLR2 agonist Pam3Cys which is a triacylated lipopeptide that has shown promise as a vaccine adjuvant acting through similar but not identical mechanisms as MPLA (Agrawal et al. J. Immunol. 171(10):4984-9 (2003)), and the iNKT agonist α-galactosylceramide (αGC) which is a marine sponge-derived glycolipid that has been pursued as a drug against cancer and autoimmunity, but has recently been recognized as a candidate vaccine adjuvant as well (Cerundolo et al. Nat. Rev. Immunol. 9, 28-38 (2009)).

The solid core may be synthesized using methods known in the art including without limitation solvent evaporation, nanoprecipitation, hot melt microencapsulation, solvent removal, and spray drying. Exemplary methods are described herein in the Examples as well as by Bershteyn et al., Soft Matter 4:1787-1787, 2008 and in US 2008/0014144 A1, the specific teachings of which relating to particle synthesis are incorporated herein by reference.

Briefly, the polymer (or copolymer) and lipids are dissolved in a organic solvent (e.g., dichloromethane). The lipids will typically comprise a functionalized lipid to which antigen and/or adjuvant may later be conjugated. Examples of suitable polymers and lipids, including functionalized lipids, are provided herein. In one instance, the polymer may be PLGA, and the lipids may be a phosphocholine such as DOPC, a phosphoglycerol such as DOPG, and a functionalized phosphoethanolamine such as a maleimide functionalized phosphoethanolamine. In some instances, the functionalized lipid also comprises polyethylene glycol (PEG) which may act as a spacer between the particle surface and the reactive group to which the antigen will be linked. The polymer and lipid solution is then emulsified in a water or other aqueous solution under agitation. The resultant emulsion is then stirred for a period of time to allow for solvent evaporation and the formation of solid particles having a self-assembled lipid coat.

Particles can then be separated according to size, if desired. This may be accomplished through centrifugation (or other pelleting means), as described herein. Particle size may be determined using scanning electron microscopy (SEM).

In one exemplary synthesis, particles were produced comprising poly(DL-lactide-co-glycolide) (PLGA) with a 50:50 lactide to glycolide ratio, the lipids 1,2-dioleoyl-sn-glycero-3-phosphocholine (DOPC), 1,2-dioleoyl-sn-glycero-3-phospho-(1′-rac-glycerol) (DOPG), and 1,2-distearoyl-sn-glycero-3-phosphoethanolamine-N-[maleimide(polyethylene glycol)2000] (maleimide-PEG2k-PE), MPLA, Pam3Cys, and/or αGC.

Lipid coated polymer core microparticles were formed as previously reported (Bershteyn et al. Soft Matter 4, 1787-1791 (2008)) by dissolving 80 mg of PLGA, 2.9 mg of DOPC, 0.75 mg of DOPG, and 1.4 mg of maleimide-PEG2k-PE in 5 mL dichloromethane, and emulsifying this solution in 40 mL of ultrapure water using an Ika T25 homogenizing disperser at 1200 RPM for 2 minutes. The emulsion was stirred for a period of time (e.g., overnight) in ambient conditions sufficient to evaporate solvent and form solid particles with self-assembled lipid shells. Gentle centrifugation for 1 minute at 1,100 g was used to separate the particles into two populations: microparticles with mean diameter 1.9+/−0.9 μm, and nanoparticles with mean diameter 116+/−35 nm (in one representative experiment). Sizes were determined using a JEOL 6320 Field-Emission High-Resolution SEM after vacuum-drying of particles onto silicon and coating with 100 A° gold, and verified by dynamic light scattering. In this synthesis, the majority of the homogenized material was in the form of microparticles.

Preparations of nanoparticles were prepared by dissolving 30 mg of PLGA, 1.3 mg of DOPC, 0.34 mg of DOPG, and 0.62 mg of maleimide-PEG2k-PE in 1 mL dichloromethane. This solution was sonicated on ice for 1 minute at 7 Watts following the addition of 200 uL ultrapure water as an internal aqueous phase. This water-in-oil emulsion was then emulsified into 6 mL ultrapure water by sonication for 5 minutes at 12 Watts on ice. This water-oil-water emulsion also supports encapsulation of a water-soluble cargo into the particles. The protocol is based on a more classical approach to poly(vinyl alcohol)-stabilized PLGA particles published by Wassell et al. (See Wassell et al. Colloids Surf A Physicochem. Eng. Aspects 2007: 292, 125-130.) Our adaptation of this approach uses lipids as surfactants in particle synthesis; an approach that has not been previously proposed by others.

To separate polymer-core nanoparticles from free liposomes, particles were layered over a cushion of 30% sucrose in ultrapure water and centrifuged at 13,000 g for 5 minutes. The liposome-containing solution present above the sucrose gradient was discarded, and the particles that formed a pellet below the sucrose gradient were retained.

The uniformity and lipid nanostructure of these particles was analyzed by cryogenic transmission electron microscopy (CryoTEM). Samples were embedded in ice by blotting 3 uL of particles in water on a 1.2/1.3 mm holey carbon-coated copper grid (Electron Microscopy Sciences, Hatfield, Pa.) and immediately freezing the sample in liquid ethane using a Leica plunge-freezing machine. Samples were imaged using a JEOL 2200FS transmission electron microscope at 185 mA emission current and 40,000× magnification. After nanoparticle synthesis and sucrose gradient purification, a lipid bilayer could be visualized by CryoTEM, with the inner leaflet tightly apposed against the electron-dense polymer core. After 7 days in saline solution, lipid delamination could be observed, signifying the beginnings of particle breakdown by hydrolysis. However, particles could be lyophilized in a solution of 2% sucrose and retained a tightly apposed lipid bilayer after reconstitution in saline. Besides single bilayers, we have also observed the formation of multilamellar “onion skins” and even “flower petals” of lipid on similarly synthesized particles made with variations in the lipid concentration and composition. (See Bershteyn et al. Soft Matter 4, 1787-1791 (2008).)

### Antigen and Adjuvant Incorporation

Agents such as antigens and adjuvants can be surface-displayed on the particles of the invention either by direct incorporation of an agent such as lipid-like antigen or adjuvant into the lipid bilayer coat, or by covalent conjugation via a reactive group such as a sulfhydryl group, primary amine, or reactive ester. If conjugated to a lipid bilayer component, the antigen or adjuvant may be derivatized to comprise a reactive group, for example as described in the Examples. The antigen or adjuvant or the lipid bilayer component to which either is conjugated may also be modified to comprise a spacer in order to distance the antigen or adjuvant from the lipid bilayer surface. A suitable spacer is PEG, as an example.

A variety of reactive groups may be used to conjugate the antigen and/or adjuvant to the lipid bilayer. Examples include maleimide groups and other thiol reactive groups, amino groups such as primary and secondary amines, carboxyl groups, hydroxyl groups, aldehyde groups, alkyne groups, azide groups, carbonyls, haloacetyl (e.g., iodoacetyl) groups, imidoester groups, N-hydroxysuccinimide esters, sulfhydryl groups, pyridyl disulfide groups, and the like. Those of ordinary skill in the art will be able to choose a reactive group pair for conjugating antigen and/or adjuvant to the lipid bilayer using the guidance provided herein and based on the knowledge in the art.

A variety of commercially available headgroup-functionalized lipids can be incorporated into the lipid bilayer. A suitable but non-limiting functionalized lipid is maleimide-PEG2k-PE, which displays a sulfhydryl-reactive maleimide ester at the end of a 2,000 Da polyethylene glycol chain. The following is a brief description of antigen conjugation to this functionalized lipid.

Protein antigen may be attached to particles via maleimide-PEG2k-PE. Protein antigen may be first purified (e.g., with a Detoxi-Gel endotoxin affinity column (Pierce Biotechnology, Rockford, Ill.)), then modified with the heterobifunctional cross-linker SAT(PEG)4 (Pierce Biotechnology, Rockford, Ill.) to convert lysines into maleimide-reactive sulfhydryl groups. Particles comprising the maleimide functionalized lipids were then incubated with sulfhydryl modified protein for 4 hours at room temperature before washing with sterile saline to remove unbound protein. As an example, green fluorescent protein (GFP) and fluorescein isothiocyanate (FITC)-labeled ovalbumin were coupled in this manner, and the resulting proteins were visualized on particle surfaces by confocal microscopy (data not shown).

Alternatively, agents that are lipid-like in structure, including lipid-like adjuvants, can be incorporated into the lipid bilayer. This can be achieved by addition of these agents to the external aqueous phase of particle synthesis, or by “post-insertion” of the desired lipid-like agent by mixing fully-formed particles with a dimethylsulfoxide solution of the agent.

As used herein, “linking” means two entities stably bound to one another by any physiochemical means. Any linkage known to those of ordinary skill in the art may be employed including covalent or noncovalent linkage, although covalent linkage is preferred. In some embodiments described herein, covalent linkage is achieved through the use of crosslinkers and functionalized components of the lipid bilayer.

### Antigen and Adjuvant Doses

As discussed herein, the particles of the invention allow for reduced amounts of antigen and/or adjuvants to be administered to a subject while still effecting robust and sufficient immune responses in the subject. The ability to achieve robust immune responses using low doses of antigen is useful in situations where antigen supply is limited, such as may occur in a pandemic situation or during the annual flu season. The ability to achieve robust immune responses using low doses of adjuvant is useful because it can reduce or eliminate unwanted side effects associated with adjuvants.

The Examples show that robust immune responses can be effected using as little as 2.5 nanograms and 10 nanograms of antigen, using OVA as a model protein antigen. This is in contrast to typical doses used to elicit antibody responses against OVA which range from one microgram to hundreds of micrograms. (See Schnare et al, Nature Immunol. 2, 947-950 (2001); Matriano et al, J. Pharm. Res, 19(1), 63-70 (2002); Fifis et al. J Immunol 173(5): 3148-3154 (2004); and Klinman et al, Vaccine 17(1) 19-25 (1999).)

Accordingly, antigen doses may be in the nanogram ranges including but not limited to 1-500 nanograms, 2 to 500 nanograms, 2 to 400 nanograms, 2 to 300 nanograms, 2 to 200 nanograms, 2 to 100 nanograms, 2 to 50 nanograms, 2 to 40 nanograms, 2 to 30 nanograms, 2 to 20 nanograms, 2 to 10 nanograms, and 2 to 5 nanograms. The bottom end of all of these ranges may also be 2.5, 3, 5 or 10 nanograms.

Adjuvant doses may be the same as or lower than those currently in use. In some instances, the dose of adjuvant may be about 75% or about 50% or as low as 10% of the amounts that are necessary in the absence of the particle formulation of the invention. In some instances, the dose of adjuvant may be 10-fold, 20-fold, 30-fold, 40-fold, 50-fold, 60-fold, 70-fold, 80-fold, 90-fold, 100-fold or more less than the amount of adjuvant in solution that is required.

### Immune Responses

The particles of the invention are used to stimulate antigen specific immune responses. Antigen specific immune responses may be antibody (or humoral) responses and/or they may be cellular responses. The antibody response may be a class switched response intending that the antibody isotype has switched from an IgD or IgM isotype to for example an IgG isotype, indicative of a mature and lasting immune response.

In some instances, sufficient immune responses may be attained using a single administration of antigen in the particulate form of the invention. In some instances, antigen doses may be decreased even further (for example into the 1-20 or 1-10 or 1-5 nanograms per dose range) provided that two or more administrations are performed.

The invention contemplates that subjects may be administered particles of the invention comprising antigen and adjuvant during a primary humoral response. Additional boosts of antigen however may be administered in a number of formulations including in solution or together with traditional adjuvants such as alum or Freund's (complete or incomplete) adjuvant in the appropriate subjects. Accordingly, the immunization protocols contemplated by the invention may comprise a prime and optionally a boost dose of antigen in the form of the particles of the invention (optionally with adjuvant incorporated therein) followed by, if necessary or desired, additional doses formulation in non-particulate form.

### Polymer Core

The particle comprises a polymer core. Preferably, the polymer core is made from biodegradable polymers which may be naturally occurring (referred to as natural) or non-naturally occurring (referred to herein as synthetic). Exemplary synthetic polymers which can be used to form the particle core include without limitation aliphatic polyesters, poly (lactic acid) (PLA), poly (glycolic acid) (PGA), co-polymers of lactic acid and glycolic acid (PLGA), polycarprolactone (PCL), polyanhydrides, poly(ortho)esters, polyurethanes, poly(butyric acid), poly(valeric acid), and poly(lactide-co-caprolactone) and exemplary natural polymers such as alginate and other polysaccharides including dextran and cellulose, collagen, chemical derivatives thereof, including substitutions, additions of chemical groups such as for example alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion. In some important embodiments, the polymer is PLGA.

### Lipids

The particles also comprise a lipid bilayer on their outermost surface. This bilayer may be comprised of one or more lipids of the same or different type. Examples include without limitation phospholipids such as phosphocholines, phosphoglycerols, phosphoethanolamines and phosphoinositols. Specific examples include without limitation DMPC, DOPC, DSPC, and various other lipids as described herein. The type, number and ratio of lipids may vary. The lipids may be isolated from a naturally occurring source or they may be synthesized apart from any naturally occurring source.

At least one (or some) of the lipids is/are amphipathic lipids, defined as having a hydrophilic and a hydrophobic portion (typically a hydrophilic head and a hydrophobic tail). The hydrophobic portion typically orients into a hydrophobic phase (e.g., within the bilayer), while the hydrophilic portion typically orients toward the aqueous phase (e.g., outside the bilayer, and possibly between adjacent apposed bilayer surfaces). The hydrophilic portion may comprise polar or charged groups such as carbohydrates, phosphate, carboxylic, sulfato, amino, sulfhydryl, nitro, hydroxy and other like groups. The hydrophobic portion may comprise apolar groups that include without limitation long chain saturated and unsaturated aliphatic hydrocarbon groups and groups substituted by one or more aromatic, cyclo-aliphatic or heterocyclic group(s). Examples of amphipathic compounds include, but are not limited to, phospholipids, aminolipids and sphingolipids.

Typically, the lipids are phospholipids. Phospholipids include without limitation phosphatidylcholine, phosphatidylethanolamine, phosphatidylglycerol, phosphatidylinositol, phosphatidylserine, and the like. It is to be understood that other lipid membrane components, such as cholesterol, sphingomyelin, cardiolipin, etc. may be used.

The lipids may be anionic and neutral (including zwitterionic and polar) lipids including anionic and neutral phospholipids. Neutral lipids exist in an uncharged or neutral zwitterionic form at a selected pH. At physiological pH, such lipids include, for example, dioleoylphosphatidylglycerol (DOPG), diacylphosphatidylcholine, diacylphosphatidylethanolamine, ceramide, sphingomyelin, cephalin, cholesterol, cerebrosides and diacylglycerols. Examples of zwitterionic lipids include without limitation dioleoylphosphatidylcholine (DOPC), dimyristoylphosphatidylcholine (DMPC), and dioleoylphosphatidylserine (DOPS). An anionic lipid is a lipid that is negatively charged at physiological pH. These lipids include without limitation phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids.

Collectively, anionic and neutral lipids are referred to herein as non-cationic lipids. Such lipids may contain phosphorus but they are not so limited. Examples of non-cationic lipids include lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, and cholesterol.

Additional nonphosphorous containing lipids include stearylamine, dodecylamine, hexadecylamine, acetyl palmitate, glycerolricinoleate, hexadecyl stereate, isopropyl myristate, amphoteric acrylic polymers, triethanolamine-lauryl sulfate, alkyl-aryl sulfate polyethyloxylated fatty acid amides, dioctadecyldimethyl ammonium bromide and the like, diacylphosphatidylcholine, diacylphosphatidylethanolamine, ceramide, sphingomyelin, cephalin, and cerebrosides. Lipids such as lysophosphatidylcholine and lysophosphatidylethanolamine may be used in some instances. Noncationic lipids also include polyethylene glycol-based polymers such as PEG 2000, PEG 5000 and polyethylene glycol conjugated to phospholipids or to ceramides (referred to as PEG-Cer).

In some instances, modified forms of lipids may be used including forms modified with detectable labels such as fluorophores. In some instances, the lipid is a lipid analog that emits signal (e.g., a fluorescent signal). Examples include without limitation 1,1′-dioctadecyl-3,3,3′,3′-tetramethylindotricarbocyanine iodide (DiR) and 1,1′-dioctadecyl-3,3,3′,3′-tetramethylindodicarbocyanine (DiD).

Preferably, the lipids are biodegradable. Biodegradable lipids include but are not limited to 1,2-dioleoyl-sn-glycero-3-phosphocholine (dioleoyl-phosphocholine, DOPC), anionic 1,2-di-(9Z-octadecenoyl)-sn-glycero-3-phospho-(1′-rac-glycerol) (dioleoyl-phosphoglycerol, DOPG), and 1,2-distearoyl-sn-glycero-3-phosphoethanolamine (distearoyl-phosphoethanolamine, DSPE). Non-lipid membrane components such as cholesterol may also be incorporated.

The lipids may be conjugated to spacers such as but not limited to PEG or they may be capable of being conjugated to such spacers, preferably at their head group.

In certain embodiments, the lipid bilayers are comprised of a functionalized lipid (as described below) and one or more non-functionalized lipids. The functionalized lipid may represent 1-100 molar percent of the lipid bilayer, including 5, 10, 15, 20, 25% or more. The non-functionalized lipids represent 1-99 molar percent of the lipid bilayer, including 75, 80, 85, 90, 95% or more. In one embodiment, the lipid bilayers are comprised of functionalized phosphoethanolamine (including maleimide functionalized phosphoethanolamine), phosphocholine and phosphoglycerol in a molar ratio of 10:72:18.

In other embodiments, the lipid bilayers may be comprised of phosphocholine and functionalized lipid in the absence of phosphoglycerol, or they may be comprised of phosphocholine and functionalized lipid with 5-50 mol % cholesterol

It is to be understood that the invention contemplates a variety of lipid bilayers as well as the use of a variety of lipid bilayer components provided such components are able to form stable bilayers.

### Functionalized Lipid Bilayer Components

At least one component of the lipid bilayer must be functionalized (or reactive). As used herein, a functionalized component is a component that comprises a reactive group that can be used to conjugate antigen and/or adjuvant. The bilayer component may be modified to comprise the reactive group.

One or more of the lipids used in the synthesis of the lipid bilayer coat of the particle may be functionalized lipids. As used herein, a functionalized lipid is a lipid having a reactive group that can be used to conjugate antigen and/or adjuvant. In some embodiments, the reactive group is one that will react with a crosslinker (or other moiety) to form crosslinks between such functionalized lipids and antigen and/or adjuvant. The reactive group may be located anywhere on the lipid that allows it to contact a crosslinker and be crosslinked to antigen and/or adjuvant. In some embodiments, it is in the head group of the lipid, including for example a phospholipid. The functionalized components (including lipids) may be conjugated to functionalized antigens and/or adjuvants in the absence or presence of a crosslinker or other reactive moiety. An example of a reactive group is a maleimide group. Maleimide groups may be conjugated to sulfhydryl containing antigens and/or adjuvants. An example of a functionalized lipid is 1,2-distearoyl-sn-glycero-3-phosphoethanolamine-N-[maleimide(polyethylene glycol)2000] (also referred to as maleimide-PEG 2k-PE). The Examples demonstrate use of this functionalized lipid in the synthesis of particles of the invention. Another example of a functionalized lipid is 1,2-dioleoyl-sn-glycero-3-phosphoethanolamine-N-[4-(p-maleimidophenyl)butyramide (also referred to as MPB). Another example of a functionalized lipid is dioleoyl-phosphatidylethanolamine 4-(N-maleimidomethyl)-cyclohexane-1-carboxylate (DOPE-mal).

It is to be understood that the invention contemplates the use of other functionalized lipids, other functionalized lipid bilayer components, other reactive groups, and other crosslinkers. In addition to the maleimide groups, other examples of reactive groups include but are not limited to other thiol reactive groups, amino groups such as primary and secondary amines, carboxyl groups, hydroxyl groups, aldehyde groups, alkyne groups, azide groups, carbonyls, haloacetyl (e.g., iodoacetyl) groups, imidoester groups, N-hydroxysuccinimide esters, sulfhydryl groups, pyridyl disulfide groups, and the like.

Functionalized and non-functionalized lipids are available from a number of commercial sources including Avanti Polar Lipids (Alabaster, Ala.).

It is to be understood that the invention contemplates various ways to link agents such as antigens and adjuvants to particles. In some instances, crosslinkers are used to effect such linkage. The invention however is not so limited. As another example, antigens and/or adjuvants may be linked to particles using click chemistry. An exemplary synthesis method uses alkyne-modified lipids and alkyne-azide chemistry. Alkyne-modified lipids can be made by mixing the lipids such as 1,2-dioleoyl-sn-glycero-3-phosphoethanolamine (DOPE, 744 mg, 1 mmol) with N-hydroxysuccinimide ester of propiolic acid (167 mg, 1 mmol) and Et₃N (202 mg, 2 mmol) in 5 mL CDCl₃.

### Crosslinkers

Crosslinkers may be used to link agents such as antigens and/or adjuvants to particles. The crosslinker may be a homobifunctional crosslinker or a heterobifunctional crosslinker, depending upon the nature of reactive groups in the lipid bilayer and the nature of the reactive group in the agent being conjugated thereto. The terms “crosslinker” and “crosslinking agent” are used interchangeably herein. Homobifunctional crosslinkers have two identical reactive groups. Heterobifunctional crosslinkers have two different reactive groups.

Various types of commercially available crosslinkers are reactive with one or more of the following groups: maleimides, primary amines, secondary amines, sulphydryls, carboxyls, carbonyls and carbohydrates. Examples of amine-specific crosslinkers are bis(sulfosuccinimidyl) suberate, bis[2-(succinimidooxycarbonyloxy)ethyl]sulfone, disuccinimidyl suberate, disuccinimidyl tartarate, dimethyl adipimate.2HCl, dimethyl pimelimidate.2HCl, dimethyl suberimidate.2HCl, and ethylene glycolbis-[succinimidyl-[succinate]]. Crosslinkers reactive with sulfhydryl groups include bismaleimidohexane, 1,4-di-[3′-(2′-pyridyldithio)-propionamido)]butane, 1-[p-azidosalicylamido]-4-[iodoacetamido]butane, and N-[4-(p-azidosalicylamido)butyl]-3′-[2′-pyridyldithio]propionamide. Crosslinkers preferentially reactive with carbohydrates include azidobenzoyl hydrazine. Crosslinkers preferentially reactive with carboxyl groups include 4-[p-azidosalicylamido]butylamine. Dithiol crosslinkers such as dithiolthietol (DTT), 1,4-di-[3′-(2′-pyridyldithio)-propionamido]butane (DPDPB), and in some instances thiol containing polymers such as (PEG)-SH2 can be used to crosslink maleimide reactive groups.

Crosslinkers reactive with alkyne groups include diazides, such as 1,14-Diazido-3,6,9,12-Tetraoxatetradecane, and other groups compatible with “click” chemistry.

Heterobifunctional crosslinkers that react with amines and sulphydryls include N-succinimidyl-3-[2-pyridyldithio]propionate, succinimidyl[4-iodoacetyl]aminobenzoate, succinimidyl 4-[N-maleimidomethyl]cyclohexane-1-carboxylate, m-maleimidobenzoyl-N-hydroxysuccinimide ester, sulfosuccinimidyl 6-[3-[2-pyridyldithio]propionamido]hexanoate, and sulfosuccinimidyl 4-[N-maleimidomethyl]cyclohexane-1-carboxylate. Heterobifunctional cross-linkers that react with carboxyl and amine groups include 1-ethyl-3-[3-dimethylaminopropyl]-carbodiimide hydrochloride. Heterobifunctional crosslinkers that react with carbohydrates and sulfhydryls include 4-[N-maleimidomethyl]-cyclohexane-1-carboxylhydrazide.2HCl, 4-(4-N-maleimidophenyl)-butyric acid hydrazide.2HCl, and 3[2-pyridyldithio]propionyl hydrazide. Other crosslinkers are bis-[(β-4-azidosalicylamido)ethyl]disulfide and glutaraldehyde.

PEGylation

The particles may be further modified by surface PEGylation. PEGylation is used clinically to increase the half-life of various agents including STEALTH liposomes. PEGylation may be accomplished by reacting functionalized lipids on the surface of the particles with a complementary functionalized PEG. The lipids are preferably not conjugated to PEG prior to particle synthesis, and rather PEG is conjugated to the particle external surface post-synthesis or PEG-lipid conjugates are introduced into the external membrane layer of the particles by “post-insertion” processes.

Reactive groups to be used to PEGylate the particles may be the same as those used to link agents to the particles, in which case no additional functionalized lipids (or other functionalized components) are required. As an example, if the particles comprise maleimide functionalized lipids, then the functionalized PEG may be thiol-PEG. Alternatively, the reactive groups used to conjugate PEG to the external surface may be different from those used to conjugate agent to the surface. Those of ordinary skill in the art will appreciate that other modified versions of PEG may be used depending on the nature of the reactive group in the functionalized lipid (or component) in the lipid bilayer. Suitable reactive groups include without limitation amino groups such as primary and secondary amines, carboxyl groups, sulfhydryl groups, hydroxyl groups, aldehyde groups, azide groups, carbonyls, maleimide groups, haloacetyl (e.g., iodoacetyl) groups, imidoester groups, N-hydroxysuccinimide esters, and pyridyl disulfide groups.

### Agents

The invention contemplates the delivery, including in some instances sustained delivery, of agents to regions, tissues or cells in vivo or in vitro using the particles of the invention. Typically the particles will comprise one or more antigens and one or more adjuvants. The particles may also contain other agents. As used herein, an agent is any atom or molecule or compound that can be used to provide benefit to a subject (including without limitation prophylactic or therapeutic benefit) or that can be used for diagnosis and/or detection (for example, imaging) in vivo or that has use in in vitro applications.

Any agent may be delivered using the particles of the invention and methods of the invention provided that it can be conjugated to, inserted in, encapsulated by, or otherwise carried by the particles of the invention. Agents, including antigens and adjuvants, may be conjugated to the external surface of the lipid bilayer, incorporated or inserted into the lipid bilayer, and/or present in the polymer core. The agent should be able to withstand the synthesis and optionally storage conditions for these particles. The particles may be synthesized and stored in, for example, an aqueous buffer at 4° C. The particles may also be stored in a lyophilized form, with a suitable excipient such as sucrose.

The agent may be without limitation a protein, a polypeptide, a peptide, a nucleic acid, a small molecule (e.g., chemical, whether organic or inorganic) drug, a virus-like particle, a steroid, a proteoglycan, a lipid, a carbohydrate, and analogs, derivatives, mixtures, fusions, combinations or conjugates thereof. The agent may be a prodrug that is metabolized and thus converted in vivo to its active (and/or stable) form. In some instances, the agents, particularly those that may be located in the polymer core, are water soluble.

The agents may be naturally occurring or non-naturally occurring. Naturally occurring agents are those that normally exist nature. Non-naturally occurring are those that do not exist in nature normally, whether produced by plant, animal, microbe or other living organism.

One class of agents is peptide-based agents such as (single or multi-chain) proteins and peptides. Examples include antibodies, single chain antibodies, antibody fragments, enzymes, co-factors, receptors, ligands, transcription factors and other regulatory factors, some antigens (as discussed below), cytokines, chemokines, and the like. These peptide-based agents may or may not be naturally occurring but they are capable of being synthesized within the subject, for example, through the use of genetically engineered cells.

Another class of agents that can be delivered using the particles of the invention includes those agents that are not peptide-based. Examples include chemical compounds that are non-naturally occurring, or chemical compounds that are not naturally synthesized by mammalian (and in particular human) cells.

A variety of agents that are currently used for therapeutic or diagnostic purposes can be delivered according to the invention and these include without limitation imaging agents, immunomodulatory agents such as immunostimulatory agents and immunoinhibitory agents, antigens, adjuvants, cytokines, chemokines, anti-cancer agents, anti-infective agents, nucleic acids, antibodies or fragments thereof, fusion proteins such as cytokine-antibody fusion proteins, Fc-fusion proteins, and the like.

Immunostimulatory Agents. As used herein, an immunostimulatory agent is an agent that stimulates an immune response (including enhancing a pre-existing immune response) in a subject to whom it is administered, whether alone or in combination with another agent. Examples include antigens, adjuvants (e.g., TLR ligands such as imiquimod and resiquimod and other imidazoquinolines, nucleic acids comprising an unmethylated CpG dinucleotide, monophosphoryl lipid A (MPLA) or other lipopolysaccharide derivatives, single-stranded or double-stranded RNA, flagellin, muramyl dipeptide), cytokines including interleukins (e.g., IL-2, IL-7, IL-15 (or superagonist/mutant forms of these cytokines), IL-12, IFN-gamma, IFN-alpha, GM-CSF, FLT3-ligand, etc.), immunostimulatory antibodies (e.g., anti-CTLA-4, anti-CD28, anti-CD3, or single chain/antibody fragments of these molecules), and the like.

Antigens. The antigen may be without limitation a cancer antigen, a self or autoimmune antigen, a microbial antigen, an allergen, or an environmental antigen. The antigen may be peptide, lipid, or carbohydrate in nature, but it is not so limited.

Cancer Antigens. A cancer antigen is an antigen that is expressed preferentially by cancer cells (i.e., it is expressed at higher levels in cancer cells than on non-cancer cells) and in some instances it is expressed solely by cancer cells. The cancer antigen may be expressed within a cancer cell or on the surface of the cancer cell. The cancer antigen may be MART-1/Melan-A, gp100, adenosine deaminase-binding protein (ADAbp), FAP, cyclophilin b, colorectal associated antigen (CRC)—0017-1A/GA733, carcinoembryonic antigen (CEA), CAP-1, CAP-2, etv6, AML1, prostate specific antigen (PSA), PSA-1, PSA-2, PSA-3, prostate-specific membrane antigen (PSMA), T cell receptor/CD3-zeta chain, and CD20. The cancer antigen may be selected from the group consisting of MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-C5). The cancer antigen may be selected from the group consisting of GAGE-1, GAGE-2, GAGE-3, GAGE-4, GAGE-5, GAGE-6, GAGE-7, GAGE-8, GAGE-9. The cancer antigen may be selected from the group consisting of BAGE, RAGE, LAGE-1, NAG, GnT-V, MUM-1, CDK4, tyrosinase, p53, MUC family, HER2/neu, p21ras, RCAS1, α-fetoprotein, E-cadherin, α-catenin, β-catenin, γ-catenin, p120ctn, gp100^{Pmel117}, PRAME, NY-ESO-1, cdc27, adenomatous polyposis coli protein (APC), fodrin, Connexin 37, Ig-idiotype, p15, gp75, GM2 ganglioside, GD2 ganglioside, human papilloma virus proteins, Smad family of tumor antigens, lmp-1, P1A, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, CD20, and c-erbB-2.

Cancer or tumor antigens can also be classified according to the cancer or tumor they are associated with (i.e., expressed by). Cancers or tumors associated with tumor antigens include acute lymphoblastic leukemia (etv6; aml1; cyclophilin b), B cell lymphoma (Ig-idiotype); Burkitt's (Non-Hodgkin's) lymphoma (CD20); glioma (E-cadherin; α-catenin; β-catenin; γ-catenin; p120ctn), bladder cancer (p21ras), biliary cancer (p21ras), breast cancer (MUC family; HER2/neu; c-erbB-2), cervical carcinoma (p53; p21ras), colon carcinoma (p21ras; HER2/neu; c-erbB-2; MUC family), colorectal cancer (Colorectal associated antigen (CRC)—0017-1A/GA733; APC), choriocarcinoma (CEA), epithelial cell-cancer (cyclophilin b), gastric cancer (HER2/neu; c-erbB-2; ga733 glycoprotein), hepatocellular cancer (α-fetoprotein), Hodgkin's lymphoma (lmp-1; EBNA-1), lung cancer (CEA; MAGE-3; NY-ESO-1), lymphoid cell-derived leukemia (cyclophilin b), melanoma (p15 protein, gp75, oncofetal antigen, GM2 and GD2 gangliosides), myeloma (MUC family; p21ras), non-small cell lung carcinoma (HER2/neu; c-erbB-2), nasopharyngeal cancer (lmp-1; EBNA-1), ovarian cancer (MUC family; HER2/neu; c-erbB-2), prostate cancer (Prostate Specific Antigen (PSA) and its immunogenic epitopes PSA-1, PSA-2, and PSA-3; PSMA; HER2/neu; c-erbB-2), pancreatic cancer (p21ras; MUC family; HER2/neu; c-erbB-2; ga733 glycoprotein), renal (HER2/neu; c-erbB-2), squamous cell cancers of cervix and esophagus (viral products such as human papilloma virus proteins and non-infectious particles), testicular cancer (NY-ESO-1), T cell leukemia (HTLV-1 epitopes), and melanoma (Melan-A/MART-1; cdc27; MAGE-3; p21ras; gp100^{Pmel1117}).

Microbial Antigens. Microbial antigens are antigens derived from microbial species such as without limitation bacterial, viral, fungal, parasitic and mycobacterial species. As such, microbial antigens include bacterial antigens, viral antigens, fungal antigens, parasitic antigens, and mycobacterial antigens. Examples of bacterial, viral, fungal, parasitic and mycobacterial species are provided herein. The microbial antigen may be part of a microbial species or it may be the entire microbe.

In one embodiment, the bacterial antigen is derived from a bacterial species selected from the group consisting of E. coli, Staphylococcal, Streptococcal, Pseudomonas, Clostridium difficile, Legionella, Pneumococcus, Haemophilus, Klebsiella, Enterobacter, Citrobacter, Neisseria, Shigella, Salmonella, Listeria, Pasteurella, Streptobacillus, Spirillum, Treponema, Actinomyces, Borrelia, Corynebacterium, Nocardia, Gardnerella, Campylobacter, Spirochaeta, Proteus, Bacteriodes, H. pylori, and anthrax.

In another embodiment, the viral antigen is derived from a viral species selected from the group consisting of HIV, Herpes simplex virus 1, Herpes simplex virus 2, cytomegalovirus, hepatitis A virus, hepatitis B virus, hepatitis C virus, human papilloma virus, Epstein Barr virus, rotavirus, adenovirus, influenza A virus, respiratory syncytial virus, varicella-zoster virus, small pox, monkey pox and SARS.

In yet another embodiment, the fungal antigen is derived from a fungal species that causes an infection selected from the group consisting of candidiasis, ringworm, histoplasmosis, blastomycosis, paracoccidioidomycosis, crytococcosis, aspergillosis, chromomycosis, mycetoma infections, pseudallescheriasis, and tinea versicolor infection.

In still another embodiment, the parasitic antigen is derived from a parasite species selected from the group consisting of amebiasis, Trypanosoma cruzi, Fascioliasis, Leishmaniasis, Plasmodium, Onchocerciasis, Paragonimiasis, Trypanosoma brucei, Pneumocystis, Trichomonas vaginalis, Taenia, Hymenolepsis, Echinococcus, Schistosomiasis, neurocysticercosis, Necator americanus, and Trichuris trichuria.

The mycobacterial antigen may be derived from a mycobacterial species such as M. tuberculosis and M. leprae, but is not so limited.

The invention intends to embrace various antigens from the infectious pathogens recited herein.

Allergens. An allergen is an agent that can induce an allergic or asthmatic response in a subject. Allergens include without limitation pollens, insect venoms, animal dander dust, fungal spores and drugs (e.g. penicillin). Examples of natural, animal and plant allergens include but are not limited to proteins specific to the following genera: Canine (Canis familiaris); Dermatophagoides (e.g. Dermatophagoides farinae); Felis (Felis domesticus); Ambrosia (Ambrosia artemiisfolia; Lolium (e.g. Lolium perenne or Lolium multiflorum); Cryptomeria (Cryptomeria japonica); Alternaria (Alternaria alternata); Alder; Alnus (Alnus gultinoasa); Betula (Betula verrucosa); Quercus (Quercus alba); Olea (Olea europa); Artemisia (Artemisia vulgaris); Plantago (e.g. Plantago lanceolata); Parietaria (e.g. Parietaria officinalis or Parietaria judaica); Blattella (e.g. Blattella germanica); Apis (e.g. Apis multiflorum); Cupressus (e.g. Cupressus sempervirens, Cupressus arizonica and Cupressus macrocarpa); Juniperus (e.g. Juniperus sabinoides, Juniperus virginiana, Juniperus communis and Juniperus ashei); Thuya (e.g. Thuya orientalis); Chamaecyparis (e.g. Chamaecyparis obtusa); Periplaneta (e.g. Periplaneta americana); Agropyron (e.g. Agropyron repens); Secale (e.g. Secale cereale); Triticum (e.g. Triticum aestivum); Dactylis (e.g. Dactylis glomerata); Festuca (e.g. Festuca elatior); Poa (e.g. Poa pratensis or Poa compressa); Avena (e.g. Avena sativa); Holcus (e.g. Holcus lanatus); Anthoxanthum (e.g. Anthoxanthum odoratum); Arrhenatherum (e.g. Arrhenatherum elatius); Agrostis (e.g. Agrostis alba); Phleum (e.g. Phleum pratense); Phalaris (e.g. Phalaris arundinacea); Paspalum (e.g. Paspalum notatum); Sorghum (e.g. Sorghum halepensis); and Bromus (e.g. Bromus inermis).

Adjuvants. The adjuvant may be without limitation alum (e.g., aluminum hydroxide, aluminum phosphate); saponins purified from the bark of the Q. saponaria tree such as QS21 (a glycolipid that elutes in the 21st peak with HPLC fractionation; Antigenics, Inc., Worcester, Mass.); poly[di(carboxylatophenoxy)phosphazene (PCPP polymer; Virus Research Institute, USA), Flt3 ligand, Leishmania elongation factor (a purified Leishmania protein; Corixa Corporation, Seattle, Wash.), ISCOMS (immunostimulating complexes which contain mixed saponins, lipids and form virus-sized particles with pores that can hold antigen; CSL, Melbourne, Australia), Pam3Cys, SB-AS4 (SmithKline Beecham adjuvant system #4 which contains alum and MPL; SBB, Belgium), non-ionic block copolymers that form micelles such as CRL 1005 (these contain a linear chain of hydrophobic polyoxypropylene flanked by chains of polyoxyethylene, Vaxcel, Inc., Norcross, Ga.), and Montanide IMS (e.g., IMS 1312, water-based nanoparticles combined with a soluble immunostimulant, Seppic)

Adjuvants may be TLR ligands. Adjuvants that act through TLR2 include without limitation Pam3Cys. Adjuvants that act through TLR3 include without limitation double-stranded RNA. Adjuvants that act through TLR4 include without limitation derivatives of lipopolysaccharides such as monophosphoryl lipid A (MPLA; Ribi ImmunoChem Research, Inc., Hamilton, Mont.) and muramyl dipeptide (MDP; Ribi) andthreonyl-muramyl dipeptide (t-MDP; Ribi); OM-174 (a glucosamine disaccharide related to lipid A; OM Pharma SA, Meyrin, Switzerland). Adjuvants that act through TLR5 include without limitation flagellin. Adjuvants that act through TLR7 and/or TLR8 include single-stranded RNA, oligoribonucleotides (ORN), synthetic low molecular weight compounds such as imidazoquinolinamines (e.g., imiquimod (R-837), resiquimod (R-848)). Adjuvants acting through TLR9 include DNA of viral or bacterial origin, or synthetic oligodeoxynucleotides (ODN), such as CpG ODN. Another adjuvant class is phosphorothioate containing molecules such as phosphorothioate nucleotide analogs and nucleic acids containing phosphorothioate backbone linkages.

### Subjects

The invention can be practiced in virtually any subject type that is likely to benefit from immune stimulation, and particularly an antigen specific immune responses. Such a response may be a humoral or a cellular immune response. Human subjects are preferred subjects in some embodiments of the invention. Subjects also include animals such as household pets (e.g., dogs, cats, rabbits, ferrets, etc.), livestock or farm animals (e.g., cows, pigs, sheep, chickens and other poultry), horses such as thoroughbred horses, laboratory animals (e.g., mice, rats, rabbits, etc.), and the like. Subjects also include fish and other aquatic species. The subjects may be normal subjects. Alternatively they may have or may be at risk of developing a condition that may be treated in whole or in part from immune stimulation, and particularly antigen specific immune responses. Treating a condition in whole or in part may include reducing or eliminating one or more symptoms of the condition.

Such conditions include cancer (e.g., solid tumor cancers or non-solid cancer such as leukemias), infections (including infections localized to particular regions or tissues in the body), autoimmune disorders, allergies or allergic conditions, asthma, transplant rejection, and the like.

Tests for diagnosing various of the conditions embraced by the invention are known in the art and will be familiar to the ordinary medical practitioner. These laboratory tests include without limitation microscopic analyses, cultivation dependent tests (such as cultures), and nucleic acid detection tests. These include wet mounts, stain-enhanced microscopy, immune microscopy (e.g., FISH), hybridization microscopy, particle agglutination, enzyme-linked immunosorbent assays, urine screening tests, DNA probe hybridization, serologic tests, etc. The medical practitioner will generally also take a full history and conduct a complete physical examination in addition to running the laboratory tests listed above.

A subject having a cancer is a subject that has detectable cancer cells. A subject at risk of developing a cancer is a subject that has a higher than normal probability of developing cancer. These subjects include, for instance, subjects having a genetic abnormality that has been demonstrated to be associated with a higher likelihood of developing a cancer, subjects having a familial disposition to cancer, subjects exposed to cancer causing agents (i.e., carcinogens) such as tobacco, asbestos, or other chemical toxins, and subjects previously treated for cancer and in apparent remission.

Subjects having an infection are those that exhibit one or typically more symptoms including without limitation fever, chills, myalgia, photophobia, pharyngitis, acute lymphadenopathy, splenomegaly, gastrointestinal upset, leukocytosis or leukopenia, and/or those in whom infectious pathogens or byproducts thereof can be detected.

A subject at risk of developing an infection is one that is at risk of exposure to an infectious pathogen. Such subjects include those that live in an area where such pathogens are known to exist and where such infections are common. These subjects also include those that engage in high risk activities such as sharing of needles, engaging in unprotected sexual activity, routine contact with infected samples of subjects (e.g., medical practitioners), people who have undergone surgery, including but not limited to abdominal surgery, etc.

The subject may have or may be at risk of developing an infection such as a bacterial infection, a viral infection, a fungal infection, a parasitic infection or a mycobacterial infection.

### Cancer

The invention contemplates administration of cancer antigens and adjuvants to subjects having or at risk of developing a cancer including for example a solid tumor cancer, using the particles of the invention. In addition, the subjects may be administered anti-cancer agents, including chemotherapeutics, antibody based therapeutics, hormone based therapeutics, and enzyme inhibitory agents.

The cancer may be carcinoma, sarcoma or melanoma. Carcinomas include without limitation to basal cell carcinoma, biliary tract cancer, bladder cancer, breast cancer, cervical cancer, choriocarcinoma, CNS cancer, colon and rectum cancer, kidney or renal cell cancer, larynx cancer, liver cancer, small cell lung cancer, non-small cell lung cancer (NSCLC, including adenocarcinoma, giant (or oat) cell carcinoma, and squamous cell carcinoma), oral cavity cancer, ovarian cancer, pancreatic cancer, prostate cancer, skin cancer (including basal cell cancer and squamous cell cancer), stomach cancer, testicular cancer, thyroid cancer, uterine cancer, rectal cancer, cancer of the respiratory system, and cancer of the urinary system.

Sarcomas are rare mesenchymal neoplasms that arise in bone (osteosarcomas) and soft tissues (fibrosarcomas). Sarcomas include without limitation liposarcomas (including myxoid liposarcomas and pleiomorphic liposarcomas), leiomyosarcomas, rhabdomyosarcomas, malignant peripheral nerve sheath tumors (also called malignant schwannomas, neurofibrosarcomas, or neurogenic sarcomas), Ewing's tumors (including Ewing's sarcoma of bone, extraskeletal (i.e., not bone) Ewing's sarcoma, and primitive neuroectodermal tumor), synovial sarcoma, angiosarcomas, hemangiosarcomas, lymphangiosarcomas, Kaposi's sarcoma, hemangioendothelioma, desmoid tumor (also called aggressive fibromatosis), dermatofibrosarcoma protuberans (DFSP), malignant fibrous histiocytoma (MFH), hemangiopericytoma, malignant mesenchymoma, alveolar soft-part sarcoma, epithelioid sarcoma, clear cell sarcoma, desmoplastic small cell tumor, gastrointestinal stromal tumor (GIST) (also known as GI stromal sarcoma), and chondrosarcoma.

Melanomas are tumors arising from the melanocytic system of the skin and other organs. Examples of melanoma include without limitation lentigo maligna melanoma, superficial spreading melanoma, nodular melanoma, and acral lentiginous melanoma. The cancer may be a solid tumor lymphoma. Examples include Hodgkin's lymphoma, Non-Hodgkin's lymphoma, and B cell lymphoma.

The cancer may be without limitation bone cancer, brain cancer, breast cancer, colorectal cancer, connective tissue cancer, cancer of the digestive system, endometrial cancer, esophageal cancer, eye cancer, cancer of the head and neck, gastric cancer, intra-epithelial neoplasm, melanoma neuroblastoma, Non-Hodgkin's lymphoma, non-small cell lung cancer, prostate cancer, retinoblastoma, or rhabdomyosarcoma.

### Infection

The invention contemplates administration of microbial antigens and adjuvants to subjects having or at risk of developing an infection such as a bacterial infection, a viral infection, a fungal infection, a parasitic infection or a mycobacterial infection, using the particles of the invention. The microbial antigen may be a bacterial antigen, a viral antigen, a fungal antigen, a parasitic antigen, or a mycobacterial antigen. In addition, the subjects may be administered anti-infective agents such as anti-bacterial agents, anti-viral agents, anti-fungal agents, anti-parasitic agents, and anti-mycobacterial agents.

The bacterial infection may be without limitation an E. coli infection, a Staphylococcal infection, a Streptococcal infection, a Pseudomonas infection, Clostridium difficile infection, Legionella infection, Pneumococcus infection, Haemophilus infection, Klebsiella infection, Enterobacter infection, Citrobacter infection, Neisseria infection, Shigella infection, Salmonella infection, Listeria infection, Pasteurella infection, Streptobacillus infection, Spirillum infection, Treponema infection, Actinomyces infection, Borrelia infection, Corynebacterium infection, Nocardia infection, Gardnerella infection, Campylobacter infection, Spirochaeta infection, Proteus infection, Bacteriodes infection, H. pylori infection, or anthrax infection.

The mycobacterial infection may be without limitation tuberculosis or leprosy respectively caused by the M. tuberculosis and M. leprae species.

The viral infection may be without limitation a Herpes simplex virus 1 infection, a Herpes simplex virus 2 infection, cytomegalovirus infection, hepatitis A virus infection, hepatitis B virus infection, hepatitis C virus infection, human papilloma virus infection, Epstein Barr virus infection, rotavirus infection, adenovirus infection, influenza virus infection, influenza A virus infection, H1N1 (swine flu) infection, respiratory syncytial virus infection, varicella-zoster virus infections, small pox infection, monkey pox infection, SARS infection or avian flu infection.

The fungal infection may be without limitation candidiasis, ringworm, histoplasmosis, blastomycosis, paracoccidioidomycosis, crytococcosis, aspergillosis, chromomycosis, mycetoma infections, pseudallescheriasis, or tinea versicolor infection.

The parasite infection may be without limitation amebiasis, Trypanosoma cruzi infection, Fascioliasis, Leishmaniasis, Plasmodium infections, Onchocerciasis, Paragonimiasis, Trypanosoma brucei infection, Pneumocystis infection, Trichomonas vaginalis infection, Taenia infection, Hymenolepsis infection, Echinococcus infections, Schistosomiasis, neurocysticercosis, Necator americanus infection, or Trichuris trichuria infection.

### Allergy and Asthma

The invention contemplates administration of allergens and adjuvants to subjects having or at risk of developing an allergy or asthma. In addition, the subjects may be administered other immunostimulatory agents including agents that stimulate a Th1 response, immunoinhibitory or immunosuppressant agents including agents that inhibit a Th2 response, anti-inflammatory agents, leukotriene antagonists, soluble IL-4 receptors, anti-IL-4 antibodies, IL-4 antagonists, anti-IL-5 antibodies, soluble IL-13 receptor-Fc fusion proteins, anti-IL-9 antibodies, CCR3 antagonists, CCR5 antagonists, VLA-4 inhibitors, and other downregulators of IgE such as but not limited to anti-IgE, cytokines such as Th1 cytokines such as IL-12 and IFN-gamma, steroids including corticosteroids such as prednisolone.

An allergy is an acquired hypersensitivity to an allergen. Allergic conditions include but are not limited to eczema, allergic rhinitis or coryza, hay fever, bronchial asthma, urticaria (hives) and food allergies, and other atopic conditions. Allergies are generally caused by IgE antibody generation against harmless allergens. Asthma is a disorder of the respiratory system characterized by inflammation, narrowing of the airways and increased reactivity of the airways to inhaled agents. Asthma is frequently, although not exclusively, associated with atopic or allergic symptoms.

The foregoing lists are not intended to be exhaustive but rather exemplary. Those of ordinary skill in the art will identify other examples of each condition type that are amenable to prevention and treatment using the methods of the invention.

### Immunization Schedules

The invention contemplates that immunization schedules may be reduced or shortened using the particles of the invention. As used herein, “shortening an immunization course” refers to reducing the number of antigen administrations. This is accomplished by stimulating a more robust immune response in the subject using the particles of the invention. The method may involve, in one embodiment, administering to a subject in need of immunization the particles of the invention comprising an antigen and an adjuvant in an amount effective to induce an antigen-specific immune response to the administered antigen in an immunization course, wherein the immunization course is shortened by at least one immunization. In other embodiments, the immunization course is shortened by one immunization, two immunizations, three immunizations, or more. In some embodiments, the immunization course is shortened to a single immunization with no boost doses required.

Immunizations that can be modified in this way include but are not limited to newborn immunizations for HBV; immunizations at for example two months of age for Polio, DTaP, Hib, HBV, Pneumococcus; immunizations at for example four months of age for Polio, DTaP, Hib, Pneumococcus; immunizations at for example six months of age for Polio, DTaP, Hib, HBV, Pneumococcus; immunizations at for example 12-15 months of age for Hib, Pneumococcus, MMR, Varicella; immunizations at for example 15-18 months of age for DtaP; immunizations at for example 4-6 years of age for Polio, DPT, MMR; immunizations at for example 11-12 years of age for MMR; immunizations at for example 14-16 years of age for tetanus-diphtheria (i.e., Td) (with a repeat as a booster every 10 years). As an example, a recommended immunization course for tetanus/diphtheria includes a primary immunization series given in adults if not received as a child, followed by routine booster doses of tetanus-diphtheria (Td) every 10 years. The method of the invention may in some instances obviate the need for booster shoots later on. As another example, hepatitis immunization commonly requires three administrations spaced at least two weeks, and sometimes one month, apart in order to develop full immunity. Using the methods of the invention, it is possible to reduce the number of injections from three to two or one. Immunization courses that can be shortened by the method of the invention include but are not limited to: HBV: Hepatitis B vaccine (3 total doses currently recommended); Polio: Inactivated polio vaccine (4 total doses currently recommended); DTaP: Diphtheria/tetanus/acellular Pertussis (3-in-1 vaccine; 5 total doses currently recommended); Hib: Haemophilus influenzae type b conjugate vaccine (4 total doses currently recommended); Pneumococcus (Prevnar): Protects against certain forms of Strep. Pneumoniae (3 total doses recommended); MMR: measles/mumps/rubella (3-in-1 vaccine; 2 total doses recommended); Td: Adult tetanus/diphtheria (2-in-1 vaccine; for use in people over age 7).

### Effective Amounts, Regimens, Formulations

The agents, including antigens and/or adjuvants, are administered in the form of particles and in effective amounts. An effective amount is a dosage of the agent sufficient to provide a medically desirable result. The effective amount will vary with the desired outcome, the particular condition being treated or prevented, the age and physical condition of the subject being treated, the severity of the condition, the duration of the treatment, the nature of the concurrent or combination therapy (if any), the specific route of administration and like factors within the knowledge and expertise of the health practitioner. It is preferred generally that a maximum dose be used, that is, the highest safe dose according to sound medical judgment.

For example, if the subject has a tumor, an effective amount may be that amount that reduces the tumor volume or load (as for example determined by imaging the tumor). Effective amounts may also be assessed by the presence and/or frequency of cancer cells in the blood or other body fluid or tissue (e.g., a biopsy). If the tumor is impacting the normal functioning of a tissue or organ, then the effective amount may be assessed by measuring the normal functioning of the tissue or organ.

In some instances the effective amount is the amount required to lessen or eliminate one or more, and preferably all, symptoms. For example, in a subject having an allergy or experiencing an asthmatic attack, an effective amount of an agent may be that amount that lessens or eliminates the symptoms associated with the allergy or the asthmatic attack. They may include sneezing, hives, nasal congestion, and labored breathing. Similarly, in a subject having an infection, an effective amount of an agent may be that amount that lessens or eliminate the symptoms associated with the infection. These may include fever and malaise. If the agent is a diagnostic agent, an effective amount may be an amount that allows visualization of the body region or cells of interest. If the agent is an antigen, the effective amount may be that amount that triggers an immune response against the antigen and preferably provides short and even more preferably long term protection against the pathogen from which the antigen derives. It will be understood that in some instances the invention contemplates single administration of an agent and in some instances the invention contemplates multiple administrations of an agent. As an example, an antigen may be administered in a prime dose and a boost dose, although in some instances the invention provides sufficient delivery of the antigen, and optionally an adjuvant, that no boost dose is required.

The invention provides pharmaceutical compositions. Pharmaceutical compositions are sterile compositions that comprise the particles of the invention and preferably agent(s) including antigens and adjuvants, preferably in a pharmaceutically-acceptable carrier. The term “pharmaceutically-acceptable carrier” means one or more compatible solid or liquid filler, diluents or encapsulating substances which are suitable for administration to a human or other subject contemplated by the invention.

The term “carrier” denotes an organic or inorganic ingredient, natural or synthetic, with which particles and preferably agent(s) including antigen and adjuvant are combined to facilitate administration. The components of the pharmaceutical compositions are comingled in a manner that precludes interaction that would substantially impair their desired pharmaceutical efficiency.

Suitable buffering agents include acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); and parabens (0.01-0.25% w/v).

Unless otherwise stated herein, a variety of administration routes are available. The particular mode selected will depend, of course, upon the particular active agent selected, the particular condition being treated and the dosage required for therapeutic efficacy. The methods provided, generally speaking, may be practiced using any mode of administration that is medically acceptable, meaning any mode that produces effective levels of a desired response without causing clinically unacceptable adverse effects. One mode of administration is a parenteral route. The term “parenteral” includes subcutaneous injections, intravenous, intramuscular, intraperitoneal, intra sternal injection or infusion techniques. Other modes of administration include oral, mucosal, rectal, vaginal, sublingual, intranasal, intratracheal, inhalation, ocular, transdermal, etc.

For oral administration, the compounds can be formulated readily by combining the particles with pharmaceutically acceptable carriers well known in the art. Such carriers enable formulation as tablets, pills, dragees, capsules, liquids, gels, syrups, slurries, films, suspensions and the like, for oral ingestion by a subject to be treated. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). Optionally the oral formulations may also be formulated in saline or buffers for neutralizing internal acid conditions or may be administered without any carriers.

Pharmaceutical preparations which can be used orally include push-fit capsules made of gelatin, as well as soft, sealed capsules made of gelatin and a plasticizer, such as glycerol or sorbitol. The push-fit capsules can contain the particles suspended in suitable liquids, such as aqueous solutions, buffered solutions, fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compositions may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount.

When it is desirable to deliver the compositions of the invention systemically, they may be formulated for parenteral administration by injection, e.g., by bolus injection or continuous infusion. Formulations for injection may be presented in unit dosage form, e.g., in ampoules or in multi-dose containers. Pharmaceutical parenteral formulations include aqueous solutions of the ingredients. Aqueous injection suspensions may contain substances which increase the viscosity of the suspension, such as sodium carboxymethyl cellulose, sorbitol, or dextran. Alternatively, suspensions of particles may be prepared as oil-based suspensions. Suitable lipophilic solvents or vehicles include fatty oils such as sesame oil, or synthetic fatty acid esters, such as ethyl oleate or triglycerides.

Alternatively, the particles may be in powder form or lyophilized form for constitution with a suitable vehicle, e.g., sterile pyrogen-free water, before use. The compositions may also be formulated in rectal or vaginal compositions such as suppositories or retention enemas, e.g., containing conventional suppository bases such as cocoa butter or other glycerides.

### Kits

The invention further contemplates kits comprising the particles of the invention. The particles may be supplied in the kit comprising one or more antigens and/or one or more adjuvants. Alternatively the kit may comprise particles having a polymer core and a lipid bilayer comprising one or more adjuvants. Such kits and particles may then be used to conjugate an antigen thereto. The antigen(s) to be conjugated to the particles may be provided in the kit or may be supplied separately. The kit may also comprise the reagents and/or instructions required for conjugating the antigen to the particle and optionally for functionalizing the antigen. The particles of the invention may be supplied in various forms depending on the type of functionalized lipid bilayer component, on the type of adjuvant, on the type of core polymer, on the type of agent present in the polymer core (if any), and the like. The particles may be provided in a buffer or in a lyophilized form.

### In Vitro Uses

The invention further contemplates that the particles of the invention can be used in vitro in a number of applications including to stimulate (or activate) antigen presenting cells such as dendritic cells. The particles may also be used in animal models. The results of such in vitro and animal in vivo uses of the particles of the invention may inform their in vivo use in humans or they may be valuable independent of any human therapeutic or prophylactic use. For example, the particles may be used to generate antibodies in mice or rabbits and those antibodies may be used as research tools for screening and the like.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### Examples

The Examples report on in vivo testing of the lipid-coated particles of the invention for delivery of protein antigens with or without co-delivered danger signals displayed in a native lipid context. The model protein antigen ovalbumin (OVA) was conjugated to PEGylated lipids incorporated in the particle lipid shells. Lipophilic adjuvants, such as monophosphoryl lipid A (MPLA) and α-galactosylceramide (αGC), were also incorporated into the surface bilayers of these particles. MPLA is a nontoxic lipopolysaccharide derivative that binds to Toll-like receptor 4 (TLR4) and is already in use in human vaccines including the papillomavirus vaccine CERVARIX™ recently approved in the United States [25]. αGC is a synthetic glycolipid that can be loaded into non-classical MHC CD1d molecules by antigen presenting cells. αGC/CD1d complexes stimulate invariant natural killer T cells (NKT cells) through their conserved T-cell receptors [26]. αGC is in clinical development as a drug against cancer and autoimmunity, but has been recognized as a candidate vaccine adjuvant as well, in part due to the recently discovered role for NKT cells in promoting humoral immune responses [17]. This is believed to be the first report of a similar formulation and use of αGC.

### Materials and Methods

Materials. PLGA with a 50:50 lactide:glycolide ratio was purchased from Lakeshore Biomaterials (Birmingham, Ala.). The lipids 1,2-dioleoyl-sn-glycero-3-phosphocholine (DOPC), 1,2-dioleoyl-sn-glycero-3-phospho-(1′-rac-glycerol) (DOPG), and 1,2-distearoyl-sn-glycero-3-phosphoethanolamine-N-[maleimide(polyethylene glycol)2000] (mal-PEG2k-PE) were purchased from Avanti Polar Lipids (Alabaster, Ala.). Carboxyfluorescein succinimidyl ester (CFSE) was from Invitrogen (Carlsbad, Calif.). MPLA was purchased from Sigma Aldrich (St. Louis, Mo.), rhodamine-conjugated Pam3Cys was purchased from Invivogen (San Diego, Calif.), and αGC was purchased from Toronto Research Chemicals Inc (North York, Ontario, Canada). n-succinimidyl s-acetyl(thiotetraethylene glycol) (SAT(PEG)₄) was purchased from Pierce Biotechnology. Solvents were from Sigma-Aldrich and used as received.

Lipid-enveloped particle synthesis. Lipid bilayer-enveloped microparticles were synthesized as previously reported [24]. Briefly, lipid (DOPC:DOPG:mal-PEG2k-PE 72:18:10 molar ratio) and polymer were co-dissolved in dichloromethane (DCM) and this organic phase was dispersed into distilled deionized ultrapure water (DDI water) by homogenization. After evaporation of DCM by stirring the emulsion for 12 hrs, solid PLGA particles with lipid bilayer coatings were recovered by centrifugation. Larger microspheres were separated from particles <1 μm by two sequential steps of centrifugation for 1 minute at 1,100 RCF. To prepare lipid-enveloped nanoparticles, we adapted a procedure published by Wassel et al. for the synthesis of poly(vinyl alcohol)-stabilized PLGA particles [37]. PLGA (30 mg) was co-dissolved in 1 mL DCM with 1.3 mg of DOPC, 0.34 mg of DOPG, and 0.62 mg of mal-PEG2k-PE to form the organic phase. An internal aqueous phase of 200 μL DDI water was dispersed in the organic phase by sonication for 1 minute on ice using a Misonix XL2000 Probe Tip Sonicator (Farmingdale, N.Y.) at 7 Watts output power. The resulting solution was immediately dispersed in 6 mL DDI water by sonication for 5 minutes on ice using the Misonix XL2000 at 12 Watts output power. DCM was evaporated overnight at ambient temperature and pressure while agitating the solution on an orbital shaker.

To purify polymer-core nanoparticles from free liposomes, particles were layered over a cushion of 30% sucrose in ultrapure water and centrifuged at 13,000 g for 5 minutes. The liposome-containing solution retained above the sucrose gradient was discarded, and the particles that formed a pellet below the sucrose gradient were retained. Self-assembly of lipids on particle surfaces was confirmed using electron microscopy. Particle size was determined using a Horiba Partica LA-950V2 Laser Diffraction Particle Size Analysis System, and confirmed using scanning electron microscopy and optical microscopy of microparticles.

Antigen conjugation to lipid-enveloped particles. To load lipid-enveloped particles with surface-displayed antigens, thiolated proteins were linked to the lipid surfaces via the maleimide terminus of mal-PEG2k-PE. As a model protein antigen, purified ovalbumin (OVA, Worthington Biochemical, Lakewood, N.J.) was passed through a Detoxi-Gel endotoxin removal affinity column (Pierce Biotechnology, Rockford, Ill.), and the resulting protein solution contained no endotoxin detectable by the Limulus Amebocyte Lysate assay (Lonza, Basel, Switzerland). OVA was modified with the heterobifunctional cross-linker SAT(PEG)₄ (Pierce Biotechnology, Rockford, Ill.) by adding a 10-fold molar excess of the crosslinker (2.2 mM) to OVA solution (0.22 mM or 10 mg/mL) and incubating on a revolving rotator for 30 minutes at room temperature. To quench NHS groups on unreacted SAT(PEG)₄ molecules, 25 mM glycine was added, and protein was incubated for an additional 15 minutes rotating at room temperature. Quenched SAT(PEG)₄ was removed by buffer exchange with a 7000 MWCO desalting spin column (Pierce Biotechnology Rockford, Ill.) and stored for up to 16 hours at 4° C. Sulfhydryl groups on SAT(PEG)₄-modified OVA were deprotected by adding 50 mM hydroxylamine and 2.5 mM EDTA (pH=7.4) and rotating for 2 hours at room temperature followed by a second buffer exchange into 10 mM EDTA (pH=7.4). Particles (70 mg/mL) were then incubated with protein (5 mg/mL) for 4 hrs at 25° C. before washing with sterile saline to remove unbound antigen. Buffers and products of the synthesis contained no detectable endotoxin. An analogous procedure was used to couple green fluorescent protein (GFP) or fluorescein isothiocyanate (FITC)-labeled OVA, and the resulting particles were visualized using a Zeiss LSM510 confocal fluorescence microscope. The particles were generally used within 12 hrs after preparation, and were stored at 4° C. until use.

The dose of protein carried by lipid-enveloped particles was determined by several independent experiments: (i) Microparticles were analyzed by flow cytometry using Reference Standard Microparticles (Bangs Labs, Fishers, Ind.) to estimate the number of protein molecules carried by each particle. (ii) Additionally, protein was stripped from particles using 1% Tween-20 or Triton X-100 and the released protein was quantitated by direct fluorescence measurements (in the case of fluorescent protein) or by enzyme-linked immunosorbent assay (ELISA). (iii) A bicinchoninic acid (BCA) protein assay (Sigma-Aldrich, St. Louis, Mo.) comparing OVA-conjugated and blank particles stripped with Tween served as a third independent test of protein dose. Because microparticles were large enough to be counted by optical microscopy, the dose of OVA measured by ELISA could also be translated into a per-particle protein quantity.

Post-insertion of lipophilic adjuvants into particle membranes. To incorporate adjuvants in the antigen-bearing particles, lipophilic Toll-like receptor agonists or the NK T-cell agonist αGC were introduced into the particle membranes via a post-insertion method. In a typical experiment, 0.7 nanomoles of each ligand (1.3 μg MPLA, 0.6 μg αGC, and/or 1.8 μg Pam3Cys from stock solutions of 2.1 mg/mL, 1.0 mg/mL, and 2.9 mg/mL in DMSO, respectively) were added to 0.1 mg of antigen-conjugated particles in 200 μL PBS, and no additional washes were performed. This post-insertion approach allowed us to compare adjuvant-containing and adjuvant-free particles derived from a single source formulation.

In vitro bioactivity of TLR agonist-loaded particles. Bone marrow-derived dendritic cells (DCs) were prepared from C57B1/6 mice as previously described [41]. DCs at day 7 of culture in a 48-well plate containing 10⁶ BMDCs/well in 1 mL of media were pulsed overnight with lipid-enveloped PLGA nanoparticles containing 10 mole % or 1 mole % MPLA (relative to lipid), or no MPLA. The total adjuvant dose per well was 7014 MPLA in the 10% case and 7 μg MPLA in the 1% case. Control cells were given equivalent quantities of MPLA alone (70, 7, or 0 μg) in complete RPMI media. Cells were blocked with anti-mouse CD16/32 and stained with fluorescent antibodies against MHC Class II or CD80 and then analyzed by flow cytometry to detect upregulation of these maturation markers.

Responses of naïve CD4⁺ or CD8⁺ T-cells transgenically expressing T cell receptors specific for OVA-derived peptides were assessed by in vitro co-culture of OT-II or OT-I primary T-cells with particle-pulsed DCs. Primary dendritic cells were isolated from spleens of C57B1/6 mice by digesting spleens with collagenase and isolating DCs using a CD11c⁺ magnetic bead isolation kit (Miltenyi Biotec). In parallel, naïve CD4⁺ T-cells or CD8+ T-cells were isolated from OT-II or OT-I TCR-transgenic mice, respectively (Jackson Laboratories), and labeled with CFSE to trace cell division following the manufacturer's instructions [42]. OVA-loaded particles with post-inserted MPLA or soluble OVA/MPLA were added to splenic DCs (12,500 cells/well) at titrated cell:antigen ratios (starting from 40:1 particles:DC, corresponding to 6.2 μg particles), and incubated for 3 hr in a total volume of 150 μL/well at 37° C. and 5% CO₂. CFSE-labeled OT-I or OT-II cells (50,000 cells/well) were then added to DCs in a volume of 50 μL complete RPMI media. This total culture volume of 200 μL/well was incubated for 3 days at 37° C. and 5% CO₂ to allow proliferation of T-cells, and CFSE dilution was then measured by flow cytometry.

Animal studies. Female BALB/c or C57B1/6 mice 6-7 weeks of age were purchased from Jackson Laboratories and cared for under local, state, and NIH care and use guidelines. Animals were immunized subcutaneously (s.c.) at the tail base with 50 uL particles or soluble protein in sterile saline, followed by a contralateral boost of the same formulation 2 or 3 weeks later. For immunization studies, the dose of antigen per particle was fixed and dose titrations were made by injecting different numbers of particles. Experiments comparing different particle compositions generally employed a single source batch of antigen-conjugated particles to control for any possible variations in particles from batch to batch. Weekly samples of 50-80 uL of blood were obtained by retro-orbital or submandibular bleeding for analysis of serum antibody titers.

Antibody titer measurements. Total IgG titers from sera were measured using an ELISA by adsorbing OVA to flat-bottom transparent 96-well plates at room temperature overnight, blocking overnight with bovine serum albumin, adding serially-diluted serum (starting from a minimal dilution of 200×) for 2 hr, and then detecting bound OVA-specific IgG antibody using HRP-labeled anti-mouse IgG (Bio-Rad). Plates were washed between each step using 0.05% Tween-20 in PBS. HRP developed with tetramethylbenzidine was measured using a Molecular Devices SpectraMax Microplate Reader. Monoclonal mouse anti-OVA IgG₁ (clone OVA-14, Sigma-Aldrich, St. Louis, Mo.) was included as a standard reference in each assay. Endpoint titers were defined as the highest dilution at which immunized serum ELISA signal exceeded the average+2 standard deviations of pre-immune sera analyzed in parallel. To interpret our titer values in more physiological terms, we used OVA-14 as a standard to determine the concentration of OVA-specific IgG as equivalents of this monoclonal antibody.

Isotype titers from sera were measured using an ELISA with identical methods to those described above for total IgG titers except that OVA-specific IgG₁ antibody was detected using HRP-labeled goat anti-mouse IgG₁ (Alpha Diagnostics) and OVA-specific IgG_{2A} antibody was detected using HRP-labeled goat anti-mouse IgG_{2A} (Alpha Diagnostics).

The avidity of IgG responses to immunization was measured using an ELISA analysis of serum binding in the presence of urea using a commonly reported procedure from the literature [43]. Serum titer analysis was conducted in duplicate assay plates until serum adsorption was complete. At this point, one plate was incubated in the presence of 6M urea for 10 min followed by washing and detection of bound IgG on both plates as above. Avidity indices were defined as the serum dilution of urea-treated samples where the ELISA absorbance was 0.5 divided by the dilution of untreated samples giving the same absorbance.

Statistical analysis. Statistical analyses were carried out using GraphPad Prism 5.0c software. For comparisons of two samples, Student's t-test was used to determine statistical significance and a P value less than 0.05 was considered significant. One-way ANOVA was applied for comparisons of multiple groups; Two-way ANOVA was used to determine statistical significance in longitudinal studies. For ANOVA analyses, Bonferroni post-tests were used to make comparisons of individual pairs of conditions.

### Results

Synthesis of antigen- and adjuvant-displaying lipid-enveloped microparticles and nanoparticles. We recently showed that synthesis of PLGA micro- or nano-particles employing phospholipids as stabilizing agents in the emulsion process leads to the self-assembly of fluid bilayer surface coatings on these particles [24]. We hypothesized that these lipid-enveloped particles could be effective agents for vaccine delivery, by co-displaying anchored antigen and lipid-embedded adjuvant molecules together on the two-dimensionally diffusing lipid bilayer surfaces. We prepared particles where the lipid coating was comprised of mal-PEG2k-PE:DOPC:DOPG in a 10:72:18 mol ratio, and conjugated thiolated protein antigens to the particles via the maleimide-PEG tethers, followed by the introduction of lipophilic adjuvant molecules via post-insertion into the lipid coatings. By changing the lipid:polymer ratio and the method of dispersion, lipid-enveloped particles with micron or submicron size distributions were obtained, having mean diameters of 2.66±1.20 μm or 212±59.2 nm, respectively (FIG. 1Bi).

Using confocal microscopy, we directly visualized the conjugation of substantial quantities of fluorescent antigens, such as GFP (FIG. 1Bii) or fluorescent OVA (not shown). We found that lipophilic adjuvants such as Pam3Cys (Toll-like receptor 2 agonist), monophosphoryl lipid A (TLR 4 agonist), or α-galactosylceramide (αGC, an invariant NK T-cell ligand) readily incorporated into the coatings of the antigen-conjugated particles. Surface loading of these ligands achieved by self-assembly during particle synthesis (ligands co-dissolved in DCM with lipids) was indistinguishable from results obtained when the ligands were added by post-insertion (illustrated in FIG. 1Biii for fluorescently-tagged Pam3Cys); we thus used the post-insertion approach for immunization studies. The quantity of antigen conjugated to the particles was determined by solubilizing the lipid surface coating with detergents and measuring the released protein by ELISA, BCA protein assay, or direct fluorescence (for GFP). These measurements were in general agreement, and gave a typical conjugation level of 0.42±0.014 μg protein per mg microparticles, corresponding to 7×10⁴ OVA molecules per microparticle. This conjugation level was also similar to the per-particle loading measured by quantitative flow cytometry, and correlated with bright protein fluorescence that could be detected on particle surfaces by confocal microscopy (FIG. 1Bii). A key advantage of this surface antigen display strategy is the ability to perform the conjugation under mild aqueous conditions and avoid exposure of potentially fragile antigens to harsh processing conditions commonly employed for encapsulation strategies.

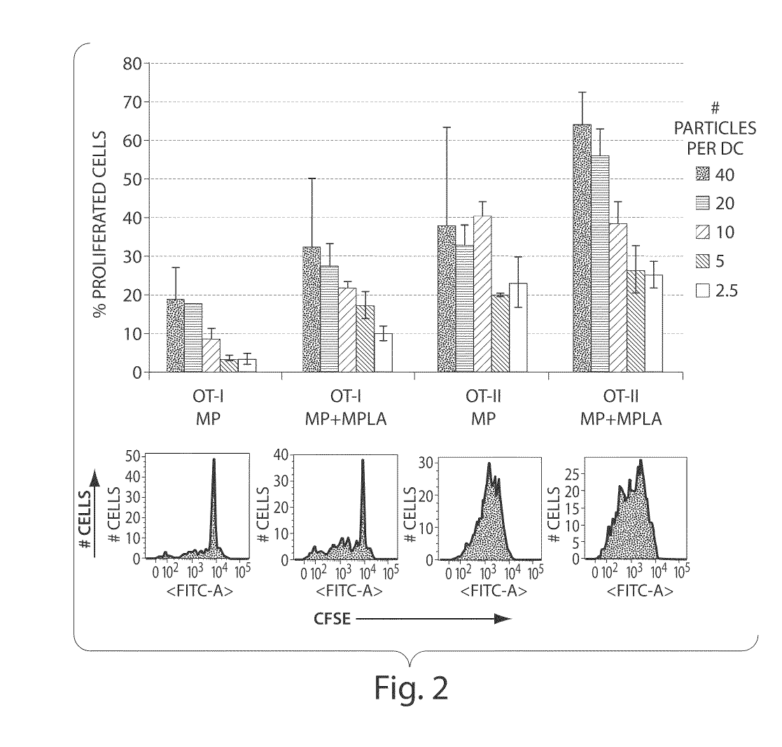

To assess the functional incorporation of adjuvant molecules in these lipid-enveloped particles and their potential for promoting cellular responses, we measured activation of dendritic cells (DCs) by MPLA-carrying nanoparticles and priming of naïve OVA-specific CD4⁺ (OT-II) or CD8⁺ (OT-I) T-cells by DCs exposed to particles or soluble ovalbumin. Bone marrow-derived DCs incubated with MPLA-decorated nanoparticles upregulated the maturation markers class II MHC and CD80 to a similar or greater extent than DCs incubated with soluble MPLA (data not shown). Notably, DCs cultured with particles lacking MPLA showed the same basal levels of MHC II/CD80 expression as cells incubated with medium alone, confirming the lack of endotoxin contamination in the materials. When primary splenic DCs were pulsed with titrated doses of OVA-conjugated particles and mixed with CFSE-labeled naïve OT-I or OT-II T-cells, T-cell proliferation was triggered in both CD4⁺ and CD8⁺ T-cells (FIG. 2), while no proliferation was observed in controls where DCs were exposed blank particles or medium (data not shown). The particles triggered cross-presentation of OVA to prime the OT-I cells at total OVA doses of only 1 ng protein per well (or less), but DCs pulsed with 10,000-fold higher doses of soluble OVA showed minimal OT-I proliferation, even in the presence of MPLA (not shown). Notably, addition of MPLA to the particles enhanced the response of both the OT-I and OT-II cells relative to particles displaying antigen alone (FIG. 2).

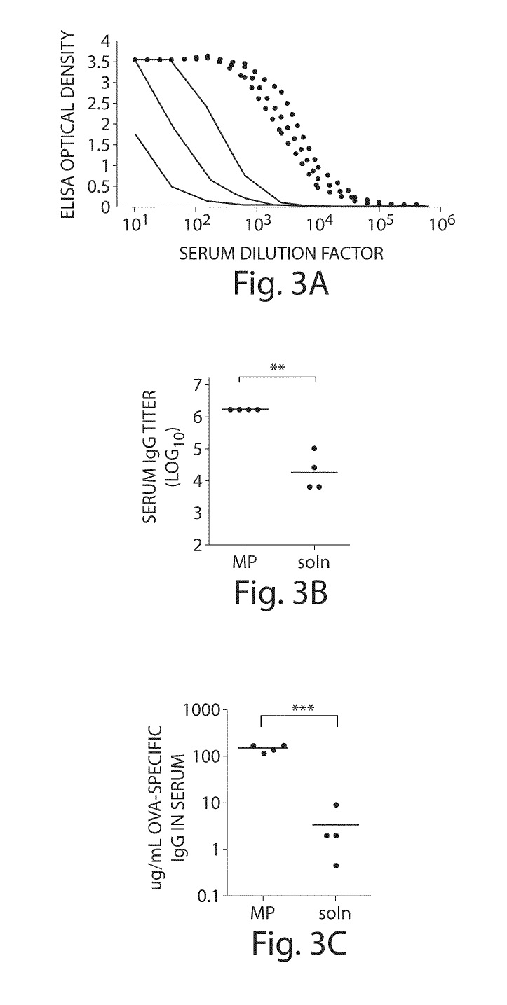

Extreme dose-sparing antibody responses elicited by particle immunization. For in vivo studies, we first confirmed that our particulate antigen delivery system could augment the serum antibody titer elicited by protein immunization. BALB/c mice were immunized s.c. with a modest dose of OVA (0.5 μg) and boosted after 3 weeks with the same dose, either in soluble form or bound to lipid-coated microparticles (1.2 mg particles/dose). OVA delivered on lipid-enveloped particles elicited substantially higher levels of serum anti-OVA IgG compared to soluble antigen, as revealed by ELISA serial dilution analysis of sera from individual immunized mice (FIG. 3A), endpoint anti-OVA IgG titers (FIG. 3B), or total anti-OVA IgG concentrations determined by calibrating against an anti-OVA monoclonal antibody standard (FIG. 3C). OVA particle vaccination generated a mean of 150 μg/mL OVA-specific IgG in serum, a 45-fold increase relative to the control soluble OVA immunization (P<0.0001). We verified that SAT(PEG)₄ modification of OVA for particle coupling had no significant influence on the protein's immunogenicity (data not shown). Thus, lipid-enveloped particle delivery of antigen substantially enhanced the humoral response at modest antigen doses.

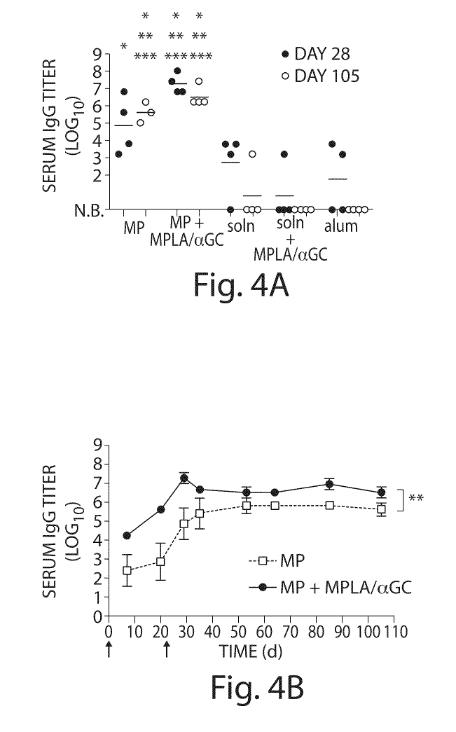

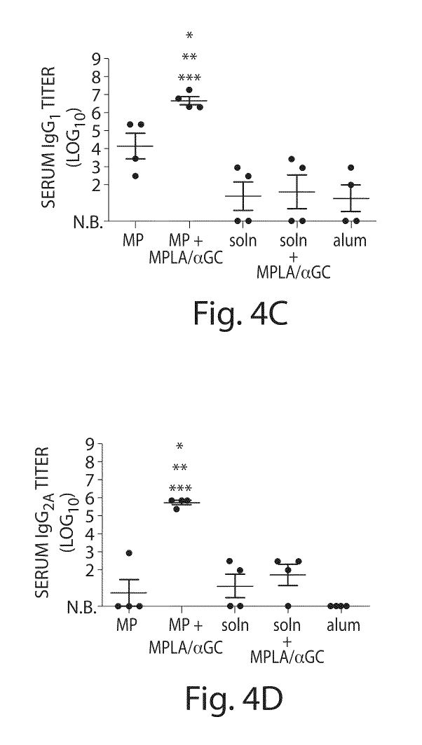

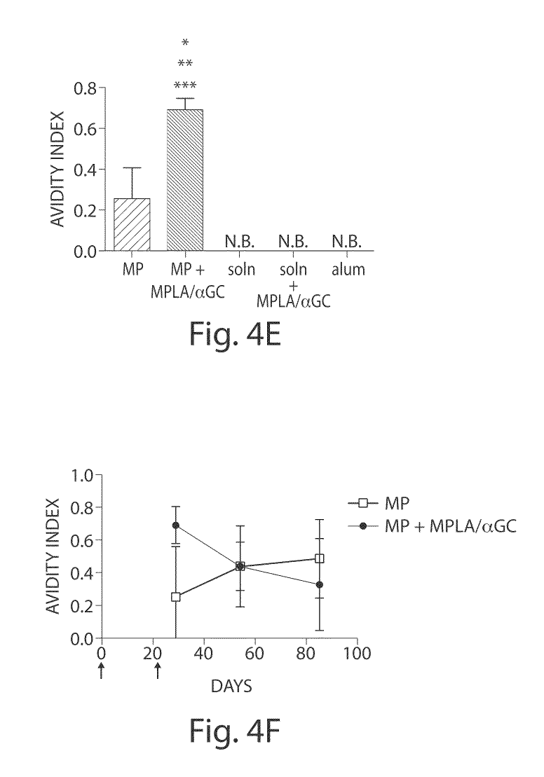

Immunization with microgram doses of protein antigens is common in murine studies [9, 12, 49], but providing saturating amounts of antigen may obscure the comparative potency of vaccines. In addition, strategies to dose-spare recombinant protein antigens are of interest for responding to both seasonal and potential pandemic diseases [29-34]. Thus, we next asked whether lipid-enveloped microparticles could potentiate antibody responses at a 50-fold lower dose of antigen, and directly compared the effectiveness of these lipid-enveloped PLGA particles with two licensed adjuvants, alum and MPLA. We found that a priming immunization with 10 ng of antigen followed by boosting with the same dose 3 weeks later elicited substantial anti-OVA IgG titers using lipid-enveloped particles as a delivery vehicle (FIG. 4A). Anti-OVA titers were increased 12-fold (P=0.0053) by co-displaying antigen together with the lipid-like adjuvants MPLA and αGC in the bilayers surrounding the particles (FIGS. 4A and B), but serum titers were maintained for at least 12 weeks regardless of whether these molecular adjuvants were included. In contrast, at this dose only a subset of mice responded when immunized with OVA solution or OVA mixed with the conventional vaccine adjuvant alum (even following boosting), and this response was not sustained in a majority of the mice (FIG. 4A). OVA mixed with soluble MPLA+αGC was also unable to elicit detectable titers at this low antigen dose (FIG. 4A). Particle immunization with or without the molecular adjuvant molecules increased IgG₁ antibody responses, but the presence of MPLA and αGC specifically boosted the Th1-like IgG_{2A} antibody response (FIG. 4C, D). Notably, measurement of the avidity of the IgG elicited by particle immunization showed that particle immunization promoted antibody responses that could still be detected following urea washes, whereas soluble OVA immunizations showed no binding under these conditions (FIG. 4E); this enhanced avidity was stable over at least 3 months post immunization (FIG. 4F). Thus, antigen delivery using lipid-coated particles was substantially more potent than either alum or soluble TLR agonist/NKT ligand for adjuvanting the humoral response, and particulate antigen delivery exhibited synergy with co-incorporated adjuvants. BALB/c and C57B1/6 mice responded with similar titers of serum IgG (data not shown). We confirmed that antibody responses elicited at such low antigen doses were not confined to OVA by repeating immunizations using GFP as an immunogen, which also elicited substantial IgG titers at doses as low as 10 ng (data not shown).

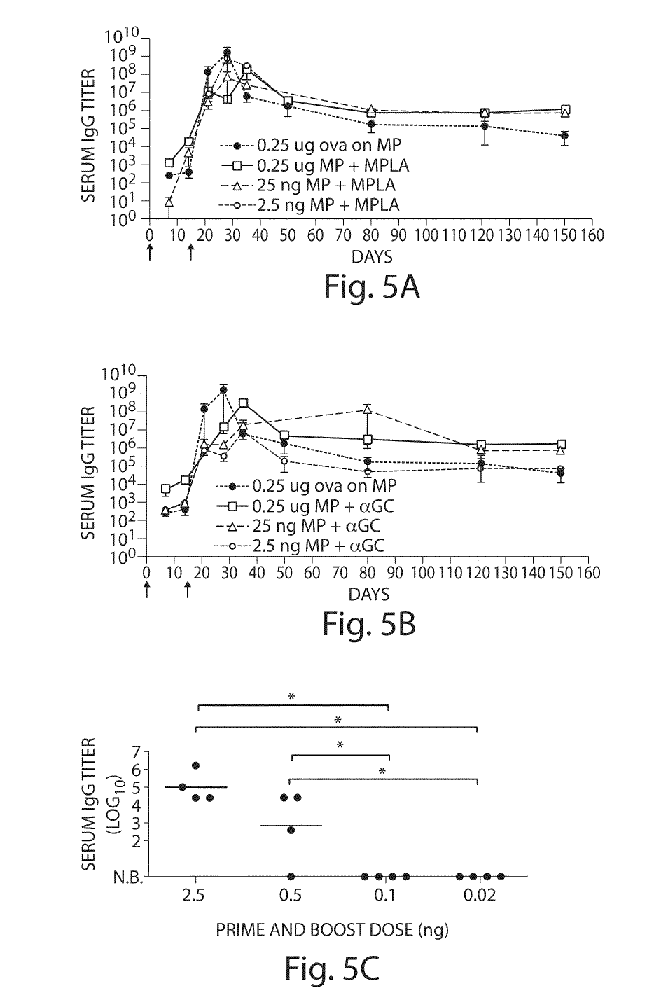

These results prompted us to explore the dose-sparing capability of lipid-enveloped particle vaccines more completely. We tested the ability of particles co-displaying antigen and individual molecular danger signals (MPLA or αGC) to elicit sustained antibody titers using antigen doses ranging from 250 ng down to 2.5 ng (FIG. 5). OVA-displaying microparticles co-delivering MPLA elevated antibody titers modestly compared to particles lacking MPLA, but this elevated antibody titer was maintained for OVA doses as low as 2.5 ng (FIG. 5A). By contrast, αGC-bearing particles induced higher titers shortly after a single immunization, but appeared to be slightly less potent in terms of inducing sustained high antibody titers, compared to particles carrying MPLA (FIG. 5B). Using MPLA-loaded particles as an optimal carrier for dose-sparing, we reduced the dose 5-fold from 2.5 ng to 0.5 ng, and only a fraction of mice had detectable OVA-specific serum IgG 2 weeks post-boost (FIG. 5C). No OVA-specific IgG was detected in mice immunized with 0.1 ng or 20 pg OVA. Thus, 2.5 ng of OVA was approximately the lowest antigen dose eliciting robust IgG responses following MPLA-adjuvanted particle delivery, under the condition used. This is 1000-fold lower than doses typically used in soluble protein immunizations.

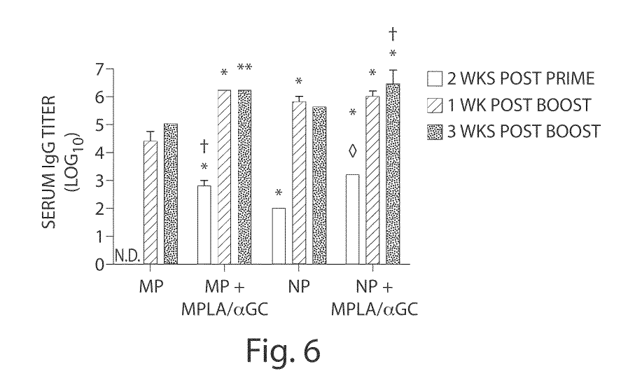

To determine whether particle size is an important parameter in this lipid-enveloped delivery system, we tested whether microparticles (mean diameter 2.66±1.20 μm) or nanoparticles (mean diameter 212±59.2 nm) were more potent in a dose-sparing immunization with 10 ng OVA. Nanoparticles consistently elicited measurable titers following a single immunization even in the absence of added danger signal molecules; however, when particles co-displayed OVA and MPLA/αGC, nanoparticles and microparticles elicited comparable antibody titers (FIG. 6). In addition, nanoparticles and microparticles elicited similar IgG1 and IgG2a titers (data not shown). Thus, in the limiting case of a single low dose without adjuvant, nanoparticles may provide a better dose-sparing vaccine delivery platform.

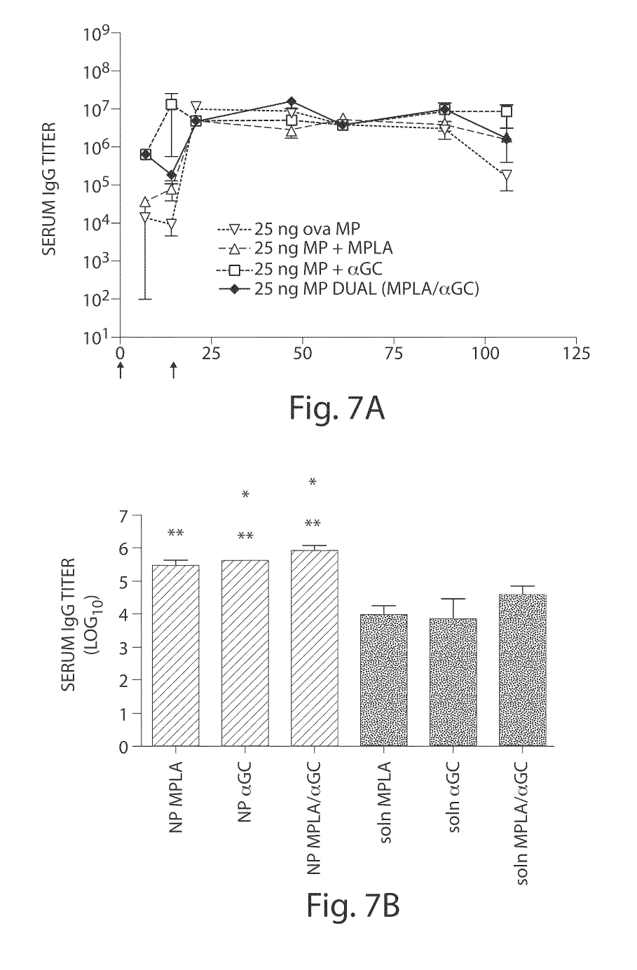

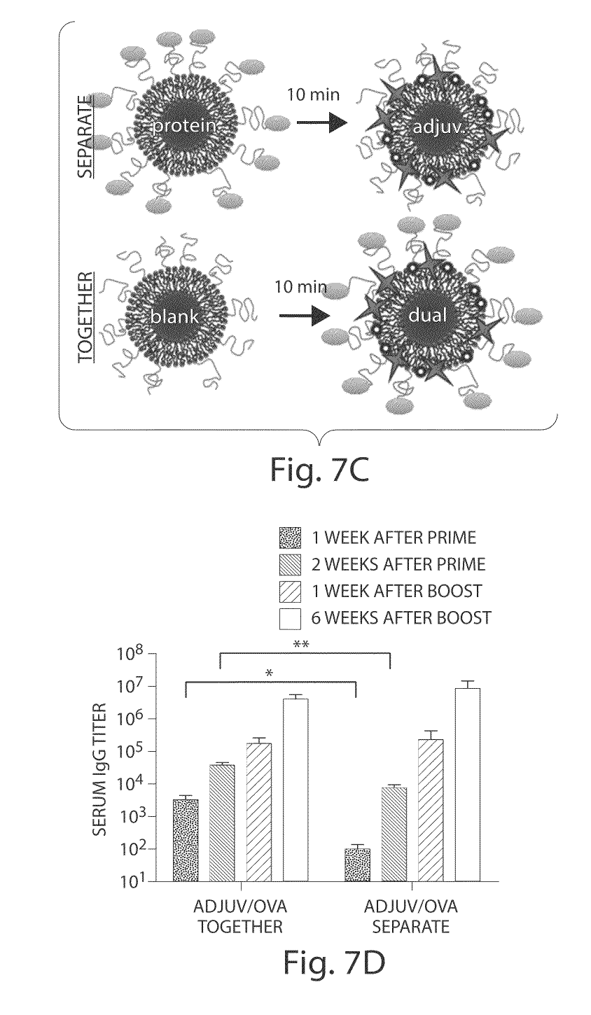

Roles for MPLA/αGC in enhancing lipid-enveloped vaccine delivery. Because MPLA and αGC promote immunity through distinct but interacting cell subsets, it has been proposed that these adjuvants could have synergistic effects [53]. Therefore, we compared the humoral responses following immunization with particles carrying MPLA alone, αGC alone, or the combination of these two ligands to determine if synergy between these adjuvants could be detected. We found that MPLA and αGC together could promote early titers comparable to αGC alone, and could sustain long-term titers at similar levels as individual adjuvants, but we did not observe further enhancement of titers above what was seen with individual adjuvant molecules (FIG. 7A). To determine whether co-loading of the two adjuvant molecules onto particles might interfere with potential synergy by directing them to the same antigen-presenting cells, we directly compared vaccination with MPLA/αGC loaded onto the antigen-bearing particles vs. the same doses of adjuvant molecules injected in soluble form, 10 minutes prior to injection of the antigen-bearing particles at the same site (FIG. 7B). MPLA/αGC co-loaded with antigen on particles showed 5-10-fold enhanced IgG titers compared to the soluble adjuvants injected separately from the antigen-loaded particles. No substantial synergy for the co-delivered adjuvants was seen compared to MPLA or αGC alone in either mode of delivery.

We next tested whether the enhancement of antibody responses elicited by MPLA/αGC in lipid-enveloped particle immunization depended on co-delivery of these adjuvant molecules on the same particle as the antigen. As illustrated schematically in FIG. 7C, mice were immunized with OVA-loaded microparticles, followed 10 minutes later by an injection of adjuvant-loaded microparticles at the same site (“separate”), to avoid lipid component exchange between particles. For particles co-displaying antigen and adjuvant molecules, we first injected blank particles, followed 10 minutes later by antigen/MPLA/αGC particles (“together”), to compare immunizations with equal total quantities of particles present. We found that prior to the booster immunization, co-display of antigen and adjuvant molecules on the same particle significantly elevated titers (34-fold increase one week after prime, P=0.028; 4.8-fold increase two weeks after prime, P=0.007)). However, no significant difference was seen after boosting (FIG. 7D). We conclude that co-delivery of antigen and adjuvant on the same particle was only important during the primary humoral response of naïve animals.

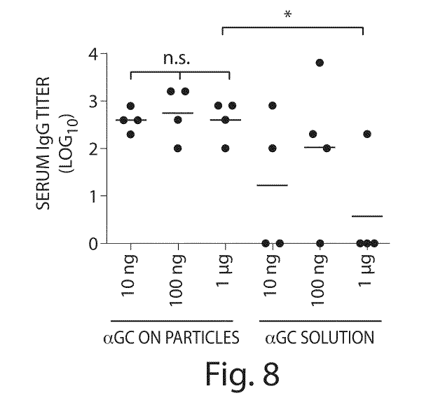

Dose sparing of molecular danger signals for antibody response by particle delivery. We finally investigated the effect of αGC dose on antibody responses to a limited dose of only 2 ng of OVA displayed on microparticles. To our knowledge, there have been no reports thus far on αGC delivery as an adjuvant for a particulate vaccine. We measured antibody titers 14 days following priming with 2 ng OVA co-loaded onto microparticles with 10 ng, 100 ng, or 1 μg αGC. Alternatively, the same dose of αGC in solution was injected 10 minutes prior to injection of particles displaying 2 ng OVA alone. The resulting IgG titers, shown in FIG. 8, revealed that all 3 doses of αGC delivered on the antigen-bearing particles elicited IgG titers from all mice following a single immunization, and the differences between groups did not reach statistical significance in any pairing of conditions (e.g., 100 ng vs. 1 μg soluble αGC: P=0.34). Notably, only a fraction of mice had detectable titers against 2 ng of antigen when αGC was delivered separately from the antigen-loaded particles in soluble form. We conclude that co-delivery of αGC with antigen together on lipid-enveloped microparticles enhances the potency of this adjuvant molecule relative to soluble delivery at the same injection site, and particle delivery further allows for at least 100-fold dose sparing of this potent immunostimulatory ligand.

### Discussion

Here we tested an approach where degradable polymer particles were “enveloped” by a functionalized phospholipid bilayer. The bilayer coating provided a facile means for anchoring antigens to the particle surfaces (via reactive lipid headgroups) and also allowed for a biomimetic presentation of membrane-incorporated adjuvant molecules. We found that particle-based delivery of vaccine antigen could significantly improve antibody responses to typical doses of protein antigen. We detected antibody by ELISA in million-fold diluted sera, corresponding to >100 μg/mL OVA-specific IgG when normalized to a commercially available monoclonal antibody standard. However, the most striking results were observed when the dose-sparing capacity of this particle-based delivery system was examined. We observed strong and sustained titers using a prime-boost regimen of a few nanograms of antigen displayed on particles. Neither the conventional adjuvant alum, nor protein solutions mixed with potent adjuvant molecules such as TLR agonists or NKT agonists, were effective at these ultra-low antigen doses.

We also observed that membrane-incorporating adjuvants co-delivered by lipid-enveloped particles could further enhance this dose-sparing capacity. Both MPLA and αGC helped to raise and maintain antibody responses, but our results suggest that each may be ideal for a different infectious disease application. MPLA-adjuvanted particles elicited lower initial titers (prior to boosting), but sustained the antibody response at the lowest doses (2.5 ng antigen) for over 150 days; such a response could help provide lifelong immunity to a disease that poses a constant hazard. By contrast, αGC-carrying particles elicited higher early titers shortly after immunization, but did not sustain long-term titers at doses as low as MPLA. Thus, αGC may be better suited in pandemic or bioterrorism scenarios in which immunity must be acquired quickly to address an immediate danger.

We initially chose to test both MPLA and αGC due to their potential for cross-talk and synergy as vaccine adjuvants [60,61]. MPLA activates dendritic cells or B-cells through Toll like receptor-4 [62], while αGC is a glycolipid that can be loaded into the cleft of non-classical CD1d MHC molecules on dendritic cells; αGC/CD1d complexes trigger activation of invariant natural killer T (iNKT) cells [60]. Recent studies have shown that iNKT cells can provide CD4 T-cell-independent help for antibody responses [17, 63, 64]. Because of this non-classical helper activity and the fact that αGC does not employ the same myd88-dependent signaling pathway used by MPLA [26,60], these adjuvants could potentially synergize in promoting vaccine responses. Indeed, Silk et al. have shown that following i.v. injection of soluble antigen, αGC, and MPLA, immune responses are amplified relative to immunizations with each of the adjuvant molecules alone with antigen [61]. However, i.v. immunization primes immune responses primarily in the spleen, and to our knowledge the same combinations have not yet been demonstrated to show synergy following traditional parenteral immunization. Here, the combination of MPLA and αGC on the same particle in s.c. immunizations did not dramatically elevate titers compared to the use of each adjuvant on its own, despite the divergent cell subsets and mechanisms through which these adjuvants act. However, combining these two ligands did allow for each feature of the response unique to the individual ligands to be achieved by a single vaccine that elicited both rapid early titer increases and sustained high titers.

Dose sparing has important practical implications, as vaccines requiring high doses of antigen suffer from high production costs and a risk of vaccine shortages [29-34]. In addition, the potency implied by highly dose-sparing formulations may be especially relevant for weakly immunogenic antigens such as recombinant HIV envelope glycoproteins, which have required high doses of antigen to elicit measurable antibody responses in animal models and human HIV vaccine trials [65-68]. In mouse models of vaccination, even highly immunogenic model antigens such as OVA are rarely used at doses below 1 μg [69-72] even in dose-response studies [73], and doses as high as 500 μg are common for model antigens [9, 12, 49]. Few studies of any vaccine have reported humoral immune responses to doses of subunit vaccine antigens as low as those reported here to our knowledge. We have also observed that particles have adjuvant-sparing capabilities in the context of humoral responses. Lipid-coated particles co-displaying antigen and αGC achieved similar titers over a wide range of doses of αGC down to 10 ng. Thus, antigen surface-display allowed for dramatic dose sparing not only of antigen, but of adjuvant as well.

We designed our study to minimize the possibility that antigen-only and adjuvant-only particles could exchange lipid molecules in solution or at the injection site by injecting blank or antigen-only particles 10 minutes prior to injection at the same site with co-loaded or adjuvant-only particles. We thus ensured that antigen and adjuvant were truly delivered on different particle populations in the test case, or co-loaded on the same particles in the control case. We found that co-loading of antigen and adjuvant was significantly advantageous after a single immunization, but that after a booster immunization, it did not matter whether antigen and adjuvant were co-loaded or delivered on separate particle populations. We further showed that αGC could be equally effective in solution injected at the same site as antigen-displaying particles. Thus, only the initial humoral response of antigen-naïve mice depended on co-loading of antigen and adjuvant.

We also found that microparticles and nanoparticles were both viable vehicles for dose-sparing delivery in a boosted vaccine regimen, although nanoparticles promoted higher titers after a single immunization, particularly in the absence of an adjuvant.

While our studies here focused on particles freshly prepared and used within ˜12 hrs, we have also shown in prior studies that lipid-enveloped PLGA micro- and nano-particles retain their original size distributions following lyophilization/reconstitution, and retain intact their surface nanoscale lipid coating organization following lyophilization/reconstitution [24]. We also confirmed in the present work that functionalized lipid headgroups remained surface-accessible following lyophilization (data not shown).

### CONCLUSION

Lipid-enveloped PLGA micro- and nano-particles were surface-modified with incorporated lipophilic molecular adjuvants and lipid-anchored protein antigens. These antigen-displaying particles elicited strong antibody titers at antigen doses of a few nanograms: far below the conventional doses used in mice, even in dose-sparing formulations such as intradermal immunizations. Co-display of adjuvants on particles further enhanced antibody responses: the TLR4 agonist MPLA sustained titers for over 150 days at the lowest doses, and the NKT agonist αGC promoted rapid IgG production after a single immunization, which may prove particularly useful in the context of a disease pandemic. The materials chosen for this vaccine platform are well suited for future clinical studies because of precedent for their use in humans (the polymer and lipid components are available from their manufacturers in GMP-compliant form), and one of the adjuvants we tested, MPLA, is already in use in human vaccines. The particles also offer the potential for controlled release of drugs from the polymer core, an aspect of significant interest for future work. Through dramatic dose sparing, this technology may facilitate protective responses with weakly immunogenic subunit vaccines, lower the cost of vaccine manufacture, and reduce the risk of seasonal or pandemic vaccine shortages.

### REFERENCES

[1] M. F. Bachmann, U. H. Rohrer, T. M. Kundig, K. Burki, H. Hengartner, R. M. Zinkernagel, The Influence of Antigen Organization on B-Cell Responsiveness, Science, 262 (1993) 1448-1451.
[2] B. Chackerian, D. R. Lowy, J. T. Schiller, Conjugation of a self-antigen to papillomavirus-like particles allows for efficient induction of protective autoantibodies, Journal of Clinical Investigation, 108 (2001) 415-423.
[3] H. M. Dintzis, R. Z. Dintzis, B. Vogelstein, Molecular Determinants of Immunogenicity—Immunon Model of Immune-Response, Proceedings of the National Academy of Sciences of the United States of America, 73 (1976) 3671-3675.
[4] A. Gamvrellis, D. Leong, J. C. Hanley, S. D. Xiang, P. Mottram, M. Plebanski, Vaccines that facilitate antigen entry into dendritic cells, Immunology and Cell Biology, 82 (2004) 506-516.
[5] M. Kovacsovicsbankowski, K. Clark, B. Benacerraf, K. L. Rock, Efficient Major Histocompatibility Complex Class-I Presentation of Exogenous Antigen Upon Phagocytosis by Macrophages, Proceedings of the National Academy of Sciences of the United States of America, 90 (1993) 4942-4946.
[6] H. Shen, A. L. Ackerman, V. Cody, A. Giodini, E. R. Hinson, P. Cresswell, R. L. Edelson, W. M. Saltzman, D. J. Hanlon, Enhanced and prolonged cross-presentation following endosomal escape of exogenous antigens encapsulated in biodegradable nanoparticles, Immunology, 117 (2006) 78-88.
[7] M. Garinot, V. Fievez, V. Pourcelle, F. Stoffelbach, A. des Rieux, L. Plapied, I. Theate, H. Freichels, C. Jerome, J. Marchand-Brynaert, Y. J. Schneider, V. Preat, PEGylated PLGA-based nanoparticles targeting M cells for oral vaccination, Journal of Controlled Release, 120 (2007) 195-204.
[8] W. L. Jiang, R. K. Gupta, M. C. Deshpande, S. P. Schwendeman, Biodegradable poly(lactic-co-glycolic acid) microparticles for injectable delivery of vaccine antigens, Advanced Drug Delivery Reviews, 57 (2005) 391-410.
[9] K. J. Maloy, A. M. Donachie, D. T. Ohagan, A. M. Mowat, Induction of Mucosal and Systemic Immune-Responses by Immunization with Ovalbumin Entrapped in Poly(Lactide-Co-Glycolide) Microparticles, Immunology, 81 (1994) 661-667.
[10] D. T. O'Hagan, M. Singh, R. K. Gupta, Poly(lactide-co-glycolide) microparticles for the development of single-dose controlled-release vaccines, Advanced Drug Delivery Reviews, 32 (1998) 225-246.
[11] S. M. Bal, B. Slutter, E. van Riet, A. C. Kruithof, Z. Ding, G. F. Kersten, W. Jiskoot, J. A. Bouwstra, Efficient induction of immune responses through intradermal vaccination with N-trimethyl chitosan containing antigen formulations, J Control Release, 142 (2010) 374-383.
[12] A. Prokop, E. Kozlov, G. Carlesso, J. M. Davidson, Hydrogel-based colloidal polymeric system for protein and drug delivery: Physical and chemical characterization, permeability control and applications, Filled Elastomers Drug Delivery Systems, 160 (2002) 119-173.
[13] J. H. Wilson-Welder, M. P. Torres, M. J. Kipper, S. K. Mallapragada, M. J. Wannemuehler, B. Narasimhan, Vaccine adjuvants: current challenges and future approaches, J Pharm Sci, 98 (2009) 1278-1316.
[14] J. E. Babensee, A. Paranjpe, Differential levels of dendritic cell maturation on different biomaterials used in combination products, Journal of Biomedical Materials Research Part A, 74A (2005) 503-510.
[15] N. L. Bennewitz, J. E. Babensee, The effect of the physical form of poly(lactic-co-glycolic acid) carriers on the humoral immune response to co-delivered antigen, Biomaterials, 26 (2005) 2991-2999.
[16] S. L. Demento, S. C. Eisenbarth, H. G. Foellmer, C. Platt, M. J. Caplan, W. M. Saltzman, I. Mellman, M. Ledizet, E. Fikrig, R. A. Flavell, T. M. Fahmy, Inflammasome-activating nanoparticles as modular systems for optimizing vaccine efficacy, Vaccine, 27 (2009) 3013-3021.
[17] P. Barral, J. Eckl-Dorna, N. E. Harwood, C. De Santo, M. Salio, P. Illarionov, G. S. Besra, V. Cerundolo, F. D. Batista, B cell receptor-mediated uptake of CD1d-restricted antigen augments antibody responses by recruiting invariant NKT cell help in vivo, Proceedings of the National Academy of Sciences of the United States of America, 105 (2008) 8345-8350.
[18] M. Friede, S. Muller, J. P. Briand, M. H. V. Vanregenmortel, F. Schuber, Induction of Immune-Response against a Short Synthetic Peptide Antigen Coupled to Small Neutral Liposomes Containing Monophosphoryl Lipid-A, Molecular Immunology, 30 (1993) 539-547.
[19] G. Kaiser-Schulz, A. Heit, L. Quintanilla-Martinez, F. Hammerschmidt, S. Hess, L. Jennen, H. Rezaei, H. Wagner, H. M. Schatzl, Polylactide-coglycolide microspheres coencapsulating recombinant tandem prion protein with CpG-oligonucleotide break self-tolerance to prion protein in wild-type mice and induce CD4 and CD8 T cell responses, Journal of Immunology, 179 (2007) 2797-2807.
[20] M. Singh, G. Ott, J. Kazzaz, M. Ugozzoli, M. Briones, J. Donnelly, D. T. O'Hagan, Cationic microparticles are an effective delivery system for immune stimulatory CpG DNA, Pharmaceutical Research, 18 (2001) 1476-1479.
[21] M. Singh, M. Briones, G. Ott, D. O'Hagan, Cationic microparticles: A potent delivery system for DNA vaccines, Proceedings of the National Academy of Sciences of the United States of America, 97 (2000) 811-816.
[22] T. Fifis, A. Gamvrellis, B. Crimeen-Irwin, G. A. Pietersz, J. Li, P. L. Mottram, I. F. C. McKenzie, M. Plebanski, Size-dependent immunogenicity: Therapeutic and protective properties of nano-vaccines against tumors, Journal of Immunology, 173 (2004) 3148-3154.
[23] S. T. Reddy, A. J. van der Vlies, E. Simeoni, V. Angeli, G. J. Randolph, C. P. O'Neill, L. K. Lee, M. A. Swartz, J. A. Hubbell, Exploiting lymphatic transport and complement activation in nanoparticle vaccines, Nature Biotechnology, 25 (2007) 1159-1164.
[24] A. Bershteyn, J. Chaparro, R. Yau, M. Kim, E. Reinherz, L. Ferreira-Moita, D. J. Irvine, Polymer-supported lipid shells, onions, and flowers, Soft Matter, 4 (2008) 1787-1791.
[25] S. Burgess, FDA Approves New Vaccine for Prevention of Cervical Cancer, in: FDA News Release, U.S. Food and Drug Administration, Oct. 16, 2009.
[26] V. Cerundolo, J. D. Silk, S. H. Masri, M. Salio, Harnessing invariant NKT cells in vaccination strategies, Nature Reviews Immunology, 9 (2009) 28-38.
[27] G. M. Glenn, M. Rao, R. L. Richards, G. R. Matyas, C. R. Alving, Murine IgG subclass antibodies to antigens incorporated in liposomes containing lipid A, Immunol Lett, 47 (1995) 73-78.
[28] H. Tamauchi, T. Tadakuma, T. Yasuda, T. Tsumita, K. Saito, Enhancement of immunogenicity by incorporation of lipid A into liposomal model membranes and its application to membrane-associated antigens, Immunology, 50 (1983) 605-612.
[29] D. S. Fedson, Preparing for pandemic vaccination: An international policy agenda for vaccine development, Journal of Public Health Policy, 26 (2005) 4-29.
[30] T. C. Germann, K. Kadau, I. M. Longini, C. A. Macken, Mitigation strategies for pandemic influenza in the United States, Proceedings of the National Academy of Sciences of the United States of America, 103 (2006) 5935-5940.
[31] R. T. Kenney, S. A. Frech, L. R. Muenz, C. P. Villar, G. M. Glenn, Dose sparing with intradermal injection of influenza vaccine, New England Journal of Medicine, 351 (2004) 2295-2301.
[32] P. K. Russell, Vaccines in civilian defense against bioterrorism, Emerging Infectious Diseases, (1999) 531-533.
[33] F. A. Sloan, S. Berman, S. Rosenbaum, R. A. Chalk, R. B. Giffin, The fragility of the US vaccine supply, New England Journal of Medicine, 351 (2004) 2443-2447.
[34] T. Yamada, Poverty, Wealth, and Access to Pandemic Influenza Vaccines., New England Journal of Medicine, 361 (2009) 1129-1131.
[35] M. Diwan, P. Elamanchili, M. Cao, J. Samuel, Dose sparing of CpG oligodeoxynucleotide vaccine adjuvants by nanoparticle delivery, Curr Drug Deliv, 1 (2004) 405-412.
[36] D. O'Hagan, M. Singh, M. Ugozzoli, C. Wild, S. Barnett, M. Chen, M. Schaefer, B. Doe, G. R. Otten, J. B. Ulmer, Induction of potent immune responses by cationic microparticles with adsorbed human immunodeficiency virus DNA vaccines, J Virol, 75 (2001) 9037-9043.
[37] R. A. Wassel, B. Grady, R. D. Kopke, K. J. Dormer, Dispersion of super paramagnetic iron oxide nanoparticles in poly(d,l-lactide-co-glycolide) microparticles, Colloids and Surfaces A: Physicochemical and Engineering Aspects, 292 (2007) 125-130.
[38] D. L. Iden, T. M. Allen, In vitro and in vivo comparison of immunoliposomes made by conventional coupling techniques with those made by a new post-insertion approach, Biochimica Et Biophysica Acta-Biomembranes, 1513 (2001) 207-216.
[39] T. Ishida, D. L. Iden, T. M. Allen, A combinatorial approach to producing sterically stabilized (Stealth) immunoliposomal drugs, Febs Letters, 460 (1999) 129-133.
[40] J. Takasaki, S. M. Ansell, Micelles as intermediates in the preparation of protein-liposome conjugates, Bioconjugate Chemistry, 17 (2006) 438-450.
[41] Y. Hori, A. M. Winans, C. C. Huang, E. M. Horrigan, D. J. Irvine, Injectable dendritic cell-carrying alginate gels for immunization and immunotherapy, Biomaterials, 29 (2008) 3671-3682.
[42] A. B. Lyons, C. R. Parish, Determination of lymphocyte division by flow cytometry, J Immunol Methods, 171 (1994) 131-137.
[43] Y. Yue, W. Xu, L. Hu, Z. Jiang, S. Xiong, Enhanced resistance to coxsackievirus B3-induced myocarditis by intranasal co-immunization of lymphotactin gene encapsulated in chitosan particle, Virology, 386 (2009) 438-447.
[44] H. Shmeeda, L. Mak, D. Tzemach, P. Astrahan, M. Tarshish, A. Gabizon, Intracellular uptake and intracavitary targeting of folate-conjugated liposomes in a mouse lymphoma model with up-regulated folate receptors, Molecular Cancer Therapeutics, 5 (2006) 818-824.
[45] C. Yoshina-Ishii, G. P. Miller, M. L. Kraft, E. T. Kool, S. G. Boxer, General method for modification of liposomes for encoded assembly on supported bilayers, Journal of the American Chemical Society, 127 (2005) 1356-1357.
[46] T. Jung, W. Kamm, A. Breitenbach, K. D. Hungerer, E. Hundt, T. Kissel, Tetanus toxoid loaded nanoparticles from sulfobutylated poly(vinyl alcohol)-graft-poly(lactide-co-glycolide): evaluation of antibody response after oral and nasal application in mice, Pharm Res, 18 (2001) 352-360.
[47] R. Shahin, M. Leef, J. Eldridge, M. Hudson, R. Gilley, Adjuvanticity and protective immunity elicited by Bordetella pertussis antigens encapsulated in poly(DL-lactide-co-glycolide) microspheres, Infect Immun, 63 (1995) 1195-1200.
[48] C. Thomasin, G. Corradin, Y. Men, H. P. Merkle, B. Gander, Tetanus toxoid and synthetic malaria antigen containing poly(lactide)/poly(lactide-co-glycolide) microspheres: Importance of polymer degradation and antigen release for immune response, Journal of Controlled Release, 41 (1996) 131-145.
[49] D. J. Campbell, E. C. Butcher, Rapid acquisition of tissue-specific homing phenotypes by CD4(+) T cells activated in cutaneous or mucosal lymphoid tissues, Journal of Experimental Medicine, 195 (2002) 135-141.
[50] H. C. Fischer, W. C. W. Chan, Nanotoxicity: the growing need for in vivo study, Current Opinion in Biotechnology, 18 (2007) 565-571.
[51] E. V. L. Grgacic, D. A. Anderson, Virus-like particles: Passport to immune recognition, Methods, 40 (2006) 60-65.
[52] M. A. Swartz, J. A. Hubbell, S. T. Reddy, Lymphatic drainage function and its immunological implications: From dendritic cell homing to vaccine design, Seminars in Immunology, 20 (2008) 147-156.
[53] M. Salio, A. O, Speak, D. Shepherd, P. Polzella, P. A. Illarionov, N. Veerapen, G. S. Besra, F. M. Platt, V. Cerundolo, Modulation of human natural killer T cell ligands on TLR-mediated antigen-presenting cell activation, Proceedings of the National Academy of Sciences of the United States of America, 104 (2007) 20490-20495.
[54] P. Thapa, G. D. Zhang, C. F. Xia, A. Gelbard, W. W. Overwijk, C. W. Liu, P. Hwu, D. Z. Chang, A. Courtney, J. K. Sastry, P. G. Wang, C. Li, D. P. Zhou, Nanoparticle formulated alpha-galactosylceramide activates NKT cells without inducing anergy, Vaccine, 27 (2009) 3484-3488.
[55] K. Lore, G. B. Karlsson Hedestam, Novel adjuvants for B cell immune responses, Curr Opin HIV AIDS, 4 (2009) 441-446.
[56] M. J. Kipper, J. H. Wilson, M. J. Wannemuehler, B. Narasimhan, Single dose vaccine based on biodegradable polyanhydride microspheres can modulate immune response mechanism, Journal of Biomedical Materials Research Part A, 76A (2006) 798-810.
[57] D. R. Burton, Antibodies, viruses and vaccines, Nature Reviews Immunology, 2 (2002) 706-713.
[58] S. A. Plotkin, Immunologic correlates of protection induced by vaccination, Pediatric Infectious Disease Journal, 20 (2001) 63-75.
[59] S. A. Plotkin, Correlates of vaccine-induced immunity, Clinical Infectious Diseases, 47 (2008) 401-409.
[60] S. Kim, S. Lalani, V. V. Parekh, L. Wu, L. Van Kaer, Glycolipid ligands of invariant natural killer T cells as vaccine adjuvants, Expert Rev Vaccines, 7 (2008) 1519-1532.
[61] J. D. Silk, I. F. Hermans, U. Gileadi, T. W. Chong, D. Shepherd, M. Salio, B. Mathew, R. R. Schmidt, S. J. Lunt, K. J. Williams, I. J. Stratford, A. L. Harris, V. Cerundolo, Utilizing the adjuvant properties of CD1d-dependent NK T cells in T cell-mediated immunotherapy, J Clin Invest, 114 (2004) 1800-1811.
[62] M. Singh, D. T. O'Hagan, Recent advances in vaccine adjuvants, Pharmaceutical Research, 19 (2002) 715-728.
[63] E. Leadbetter, M. Brigl, P. Illarionov, N. Cohen, M. Luteran, S. Pillai, G. Besra, M. Brenner, NK T cells provide lipid antigen-specific cognate help for B cells, Proceedings of the National Academy of Sciences, 105 (2008) 8339.
[64] G. Galli, P. Pittoni, E. Tonti, C. Malzone, Y. Uematsu, M. Tortoli, D. Maione, G. Volpini, O. Finco, S, Nuti, S. Tavarini, P. Dellabona, R. Rappuoli, G. Casorati, S. Abrignani, Invariant NKT cells sustain specific B cell responses and memory, Proc Natl Acad Sci USA, 104 (2007) 3984-3989.
[65] K. Banerjee, S. Andjelic, P. J. Klasse, Y. Kang, R. W. Sanders, E. Michael, R. J. Durso, T. J. Ketas, W. C. Olson, J. P. Moore, Enzymatic removal of mannose moieties can increase the immune response to HIV-1 gp120 in vivo, Virology, 389 (2009) 108-121.
[66] P. B. Gilbert, M. L. Peterson, D. Follmann, M. G. Hudgens, D. P. Francis, M. Gurwith, W. L. Heyward, D. V. Jobes, V. Popovic, S. G. Self, F. Sinangil, D. Burke, P. W. Berman, Correlation between immunologic responses to a recombinant glycoprotein 120 vaccine and incidence of HIV-1 infection in a phase 3 HIV-1 preventive vaccine trial, Journal of Infectious Diseases, 191 (2005) 666-677.
[67] B. S. Graham, M. J. McElrath, R. I. Connor, D. H. Schwartz, G. J. Gorse, M. C. Keefer, M. J. Mulligan, T. J. Matthews, S. M. Wolinsky, D. C. Montefiori, S. H. Vermund, J. S. Lambert, L. Corey, R. B. Belshe, R. Dolin, P. F. Wright, B. T. Korber, M. C. Wolff, P. E. Fast, A. V. E. Grp, H. I. P. Grp, Analysis of intercurrent human immunodeficiency virus type 1 infections in phase I and II trials of candidate AIDS vaccines, Journal of Infectious Diseases, 177 (1998) 310-319.
[68] C. Grundner, Y. X. Li, M. Louder, J. Mascola, X. Z. Yang, J. Sodroski, R. Wyatt, Analysis of the neutralizing antibody response elicited in rabbits by repeated inoculation with trimeric HIV-1 envelope glycoproteins, Virology, 331 (2005) 33-46.
[69] C. F. Anderson, D. M. Mosser, A novel phenotype for an activated macrophage: the type 2 activated macrophage, Journal of Leukocyte Biology, 72 (2002) 101-106.
[70] M. G. Bruce, A. Ferguson, The Influence of Intestinal Processing on the Immunogenicity and Molecular-Size of Absorbed, Circulating Ovalbumin in Mice, Immunology, 59 (1986) 295-300.
[71] D. M. Klinman, K. M. Barnhart, J. Conover, CpG motifs as immune adjuvants, Vaccine, 17 (1999) 19-25.
[72] M. Schnare, G. M. Barton, A. C. Holt, K. Takeda, S. Akira, R. Medzhitov, Toll-like receptors control activation of adaptive immune responses, Nature Immunology, 2 (2001) 947-950.
[73] T. Uchida, S. Martin, T. P. Foster, R. C. Wardley, S. Grimm, Dose and Load Studies for Subcutaneous and Oral Delivery of Poly(Lactide-Co-Glycolide) Microspheres Containing Ovalbumin, Pharmaceutical Research, 11 (1994) 1009-1015.
[74] D. Davis, G. Gregoriadis, Liposomes as Adjuvants with Immunopurified Tetanus Toxoid—Influence of Liposomal Characteristics, Immunology, 61 (1987) 229-234.
[75] D. Davis, A. Davies, G. Gregoriadis, Liposomes as Adjuvants with Immunopurified Tetanus Toxoid—the Immune-Response, Immunology Letters, 14 (1987) 341-348.
[76] J. Kazzaz, M. Singh, M. Ugozzoli, J. Chesko, E. Soenawan, D. T. O'Hagan, Encapsulation of the immune potentiators MPL and RC529 in PLG microparticles enhances their potency, Journal of Controlled Release, 110 (2006) 566-573.
[77] Y. S. Chen, Y. C. Hung, W. H. Lin, G. S. Huang, Assessment of gold nanoparticles as a size-dependent vaccine carrier for enhancing the antibody response against synthetic foot-and-mouth disease virus peptide, Nanotechnology, 21 (2010)-.
[78] D. F. Nixon, C. Hioe, P. D. Chen, Z. N. Bian, P. Kuebler, M. L. Li, H. Qiu, X. M. Li, M. Singh, J. Richardson, P. McGee, T. Zamb, W. Koff, C. Y. Wang, D. OHagan, Synthetic peptides entrapped in microparticles can elicit cytotoxic T cell activity, Vaccine, 14 (1996) 1523-1530.
[79] J. H. Eldridge, J. K. Staas, J. A. Meulbroek, T. R. Tice, R. M. Gilley, Biodegradable and Biocompatible Poly(D1-Lactide-Co-Glycolide) Microspheres as an Adjuvant for Staphylococcal Enterotoxin-B Toxoid Which Enhances the Level of Toxin-Neutralizing Antibodies, Infection and Immunity, 59 (1991) 2978-2986.
[80] Y. Tabata, Y. Inoue, Y. Ikada, Size effect on systemic and mucosal immune responses induced by oral administration of biodegradable microspheres, Vaccine, 14 (1996) 1677-1685.
[81] R. Nakaoka, Y. Inoue, Y. Tabata, Y. Ikada, Size effect on the antibody production induced by biodegradable microspheres containing antigen, Vaccine, 14 (1996) 1251-1256.
[82] V. Kanchan, A. K. Panda, Interactions of antigen-loaded polylactide particles with macrophages and their correlation with the immune response, Biomaterials, 28 (2007) 5344-5357.
[83] Gupta R K. Adv Drug Deliv Rev. 32(3):155-172 (1998).
[84] Fahmy et al. Biomaterials 26(28), 5727-5736 (2005).
[85] Mata-Haro et al. Science 316(5831), 1628-1632 (2007).
[86] Agrawal et al. J. Immunol. 171(10):4984-9 (2003).
[87] Matriano et al, J. Pharm. Res, 19(1), 63-70 (2002).

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A particle comprising
a biodegradable polymer core,
a lipid bilayer coat, wherein the lipid bilayer coat is conjugated to an antigen, and
an adjuvant incorporated into the lipid bilayer coat,
wherein the particle is a nano- or microparticle.

2. The particle of claim 1, wherein the biodegradable polymer is PLGA.

3. The particle of claim 1, wherein the lipid bilayer coat comprises phosphocholine.

4. The particle of claim 1, wherein the lipid bilayer coat comprises phosphoglycerol.

5. The particle of claim 1, wherein the lipid bilayer coat comprises a phosphoethanolamine.

6. The particle of claim 5, wherein the phosphoethanolamine is functionalized with a reactive group.

7. The particle of claim 6, wherein the reactive group is maleimide.

8. The particle of claim 1, wherein the antigen is conjugated to a functionalized lipid in the lipid bilayer coat.

9. The particle of claim 8, wherein the functionalized lipid is a maleimide functionalized phosphoethanolamine.

10. The particle of claim 8, wherein the antigen is a protein antigen.

11-12. (canceled)

13. The particle of claim 1, wherein the adjuvant is MPLA.

14. The particle of claim 1, wherein the adjuvant is alpha-GC.

15. A particle comprising
a PLGA polymer core,
a lipid bilayer coat comprising phosphocholine, phosphoglycerol and phosphoethanolamine, wherein the lipid bilayer coat is conjugated to an antigen, and
monophosphoryl lipid A (MPLA) incorporated into the lipid bilayer coat.

16. A particle comprising
a PLGA polymer core,
a lipid bilayer coat comprising phosphocholine, phosphoglycerol and phosphoethanolamine, wherein the lipid bilayer coat is conjugated to an antigen, and
alpha-galactosylceramide (αGC) incorporated into the lipid bilayer coat.

17. A particle comprising
a PLGA polymer core,
a lipid bilayer coat comprising phosphocholine, phosphoglycerol and phosphoethanolamine, wherein the lipid bilayer coat is conjugated to an antigen, and
adjuvant Pam3Cys incorporated into the lipid bilayer coat.

18. A particle comprising
a polymer core,
a lipid bilayer coat comprising phosphocholine, phosphoglycerol and phosphoethanolamine, wherein the lipid bilayer coat is conjugated to an antigen, and
one or more adjuvants selected from the group consisting of monophosphoryl lipid A (MPLA), alpha-galactosylceramide (αGC) and Pam3Cys incorporated into the lipid bilayer coat.

19-25. (canceled)

26. A method comprising
administering to a subject in need of immune stimulation a composition comprising the particles of claim 1 in an effective amount to stimulate an antigen specific immune response.

27-39. (canceled)

40. A method comprising
conjugating an antigen to a functionalized lipid of a particle comprising a biodegradable polymer core and a lipid bilayer coat, wherein the lipid bilayer coat comprises a lipid-like adjuvant.

41. A method comprising
conjugating an antigen to a functionalized lipid of a particle comprising a biodegradable polymer core and a lipid bilayer coat, and
incorporating into the lipid bilayer coat a lipid-like adjuvant.

42-44. (canceled)

45. A pharmaceutical composition comprising particles comprising
a biodegradable polymer core,
a lipid bilayer coat, wherein the lipid bilayer coat is conjugated to an antigen, and a pharmaceutically acceptable carrier,
wherein the antigen is present in a dose of 1-10 nanograms.

46. (canceled)
